(19) United States
(12) Patent Application Publication
(10) Pub. No.: US 2002/0064809 A1
(43) Pub. Date: May 30, 2002

(54) Focused acoustic ejection cell sorting system and method

(76) Inventors: Mitchell W. Mutz, Palo Alto, CA (US); Richard N. Ellson, Palo Alto, CA (US); David Soong-Hua Lee, Mountain View, CA (US)

Correspondence Address: REED & ASSOCIATES, 800 MENLO AVENUE, SUITE 210, MENLO PARK, CA, 94025, US

(21) Appl. No.: 09/751,666

(22) Filed: Dec. 28, 2000

Related U.S. Application Data
(63) Continuation-in-part of application No. 09/727,391, filed on Nov. 29, 2000.

(51) Int. Cl. G01N 1/30; G01N 33/48; C12N 15/01

(52) U.S. Cl. 435/40.5; 435/446

(57) ABSTRACT

A method is provided for acoustically ejecting from a container that is preferably a channel, a plurality of particles or localized volumes that can be single living cells contained in fluid droplets toward sites on a substrate surface or alternatively or in addition thereto into containers or channels for deposition at a target array site or a container or channel by acoustic ejection. An integrated cell sorting and arraying system also is provided that is capable of selective sorting, into channels or other containers substantially transected by a common plane, parallel to a surface of the fluid, transecting the container from which cells are ejected by selective ejection of cells with adjustable velocity parallel to the fluid surface, and simultaneously selectively forming an array of cells on a substrate surface comprising an array of substantially planar sites is provided, wherein each site contains a single cell. Additionally provided is a method of forming arrays of single live cells more efficiently, rapidly, flexibly and economically than by other cell array approaches, while permitting efficient, continuous and simultaneous sorting of cells based upon selection by measurement of detectible properties quantitatively or semiquantitatively, and multiple ejection target selections permitting non-binary or severally branched decision making. An integrated system and methods are also provided for ejection of selected particles or circumscribed volumes such as live cells from a continuous stream of particles or circumscribed volumes flowing in fluidic ejection channels into flowing fluidic target channels based upon selection by measurement of detectible properties quantitatively or semiquantitatively, and multiple ejection target selections permitting non-binary or severally branched decision making integrated with the measurement by way of a processor.

### CROSS-REFERENCE TO RELATED APPLICATIONS

This application is a continuation in part of U.S. patent application Ser. No. 09/727,391, filed Nov. 29, 2000 which patent application is incorporated herein by reference.

### TECHNICAL FIELD

This invention relates generally to the use of focused acoustic energy in the spatially directed ejection of cells suspended in a carrier fluid, for efficient, non-destructive and complete sorting of cells.

### BACKGROUND

The efficient, non-destructive and complete sorting of cells is important in basic biological and medical research. For example, cell sorting is commonly used in immunology, where cells displaying specific markers are segregated from other cells via an optical property such as fluorescence. Another application is medical therapeutics, where often a certain autologous or heterologous cell is desired for transplantation as in therapy for neoplasia. Advances in microfabrication of biocompatible materials and bioengineering in general suggest that more effective cell sorting methods will find use in tissue engineering applications.

Early cell sorting devices distinguished between cells based upon physical parameters. Such cell sorting techniques include filtration, which distinguishes cell size, and centrifugation, which distinguishes cell density. These methods are effective if the cell population of interest differs significantly in size or density, from the other cells in the cell mixture. However, when the individual cell populations in the cell mixture differ insufficiently in size or density, neither filtration nor centrifugation techniques can separate them effectively.

To overcome these disadvantages, techniques were developed to distinguish cell populations based on the display of surface markers or epitopes. These techniques differentiated between cell populations based on tagging elements attached to the cell surface and have become a significant cell sorting tool. Fluorescence-Activated Cell Sorting (FACS) employs a fluorescent-antibody label or tag that binds a specific cell surface marker. Although some FACS sorting operations may rely upon detected intrinsic fluorescence of a cell, e.g. an intrinsic tag, such sorters operate primarily in a binary manner, e.g whether or not a cell bears sufficient fluorescent labels for triggering a single separation threshold. The binary separation is controlled by setting a threshold (“gate”) to trigger the separative event.

Because FACS sorters examine a single cell at a time, the rate of cell separation is relatively slow. Generally, a FACS sorter can provide a cell sorting rate of 10³ cells/second. Higher cell sorting rates are possible, but higher sorting rates may damage some cells. A limited number of FACS sorters are present in many laboratories because they are costly and must be operated by skilled technicians.

Another cell tagging based separation method is known as High Gradient Magnetic Separation (HGMS). Magnetic based sorting was first employed in the mining and industrial arts, and separates using differences in intrinsic magnetic properties between the sorted materials for operation (see U.S. Pat. No. 2,056,426 to Frantz).

In HGMS, a heterogeneous cell population or cell mixture, which includes a magnetically tagged cell sub-population, passes through an applied magnetic field, and the cell sub-population labeled with the magnetic cell tags is selectively affected. The cell sub-population bearing the magnetic tags will experience a net directional magnetic force exerted by the magnetic field and often collected by adhering to the magnetic source itself, or to a cell collector near the magnetic source. Thus HGMS is also primarily binary in nature as separation is based on the presence or absence of a cell bears magnetic tags.

One shortcoming of HGMS, which can be faster than FACS, is that the cell sub-population of interest can be damaged during the HGMS process because of the magnetic force massing the cells at the collector. The HGMS process sorts cells based on a binary tagging as does the FACS system. Binary separation techniques based on a parameter such as magnetic or fluorescence properties, are important for separating cells. However a need exists for separating cells in a non-binary manner, based on the intensity of a specified parameter, such as the intensity of a detected magnetic or fluorescent signal.

Recently a system and method for sorting cells based on the amount of magnetic tags bound to the cell has been described (U.S. Pat. No. 6,120,735 to Zborowski et al.) using a channel in which the tagged cells flow through a magnetic field. The method is capable of higher throughput while maintaining comparable to higher cell viability compared to traditional FACS or HGMS. A population of particles having different magnetic susceptibilities is subjected to a magnetic field during flow to create enriched lamina. Divided flow compartments are generated within the channel to generate efferent fractionated flow streams. It will be immediately apprehended that the fractionated cell flow streams will not be absolutely purified but enriched.

Specifically the equilibrium distribution of the cells in different flow compartments in the field will depend upon the position of the flow compartment in the field according to the corresponding energy for the particle at that distance, e.g fraction in a compartment between 0 and w in the flow channel will be: f=exp(−(E(w)−E(0))/kT) where E(w) is the field potential energy as a function of w and E(0) is the lowest potential energy position, thus a more interactive particle having an energy function E₁(w) that rises more steeply from E₁(0) than a less interactive particle having an energy function E₂(w) that rises less steeply from E₂(0) (note that E₁(0) will normally be unequal to E₂(0)). Pre-equilibrium enrichment is necessarily less than that obtained at equilibrium, but at an earlier time. Statistical enrichment is a relatively less stringent separation, and the resulting fractions are less pure than the separation results obtainable by binary tagging methods. Thus the higher throughput while maintaining cell viability, of fractional enrichment methods, is obtained by a sacrifice in purity. A need therefore exists for methods of cell sorting which allow greater throughput and flexibility to perform non-binary separations without sacrificing purity.

Another recently described method for sorting cells increases throughput and avoids mere enrichment, but sacrifices cells by destroying all detected unwanted cells with a laser (U.S. Pat. No. 5,158,889 to Hirako et al., 1992).

Methods in cell sorting include the ability to separate a single file, fluidically continuous procession of cells in a channel into a fluidically discontinuous procession of individual droplets containing single cells as described in U.S. Pat. Nos. 3,710,933 to Fulwyler et al., and 3,380,584 and 4,148,718 both to Fulwyler. The procession of individual droplets is formed by vibrating a flow chamber or orifice through which the flow passes, usually at a frequency on the order of 40,000 Hz. Such droplets may be ejected from an orifice; the ejection is by manipulation of pre-formed fluidic droplets containing cells in a fluidic channel. The cells in single file are separated, resulting in a smaller number of cells passing a detection or ejection point per unit of time, thus reducing throughput and efficiency as selected cells can not be ejected at a given location from the procession in as rapid succession regardless of their location in the procession as in the case where the fluid is continuous. Also many of the inflexibilities associated with manipulating individual cells in a channel containing many cells exist. The speed of manipulating individual cells in a channel is inherently limited, for example, because the flow may need to be slowed or stopped to prevent cellular collisions during the manipulation of cells in a channel or system of interconnected channels.

One example is jet-in-air sorters, which are often optimized for commercial mammalian cell sorting. Lymphoid cells are commonly sorted and have diameters ranging from 8 to 14 μm, while spermatocytes may have a long dimension of up to 200 μm. Piezo-based jet-in-air systems must be tuned to the specific diameters of the cells to be sorted, making difficult the sorting of several subpopulations of cells having substantially different mean size. Fluidic parameters that must be changed to tune the system for a different cell size or fluid viscosity include flow tip diameter, sheath pressure, flow rate, droplet drive frequency, drive amplitude, droplet spacing, and droplet breakoff point.

Efficiency disadvantages of piezo-based systems also arise from relying on flowstreams to space out cells to prevent cell bunching in the flow stream, thus reducing the capacity to quickly locate cells for sorting operations. For example, to avoid cell bunching, one drop out of ten may contain a cell. Consequently, for a repetition rate of 32,000, only 3200 cells may be counted per second, a 10-fold lower efficiency compared to each droplet containing a cell.

A need therefore exists for a method and system capable of sorting a large range of particle sizes without requiring changing the flow tip or addressing other particle size predicated fluidic parameters. Indeed, a need exists for cell sorting methods and systems which do not require such flow tips to eliminate the potential for clogging. A need exists for a sorting system and method that can readily discriminate between clumps of cells and single cells without clogging, permitting clumps to be identified and sorted separately. A need also exists for a sorting system and method that permits adjustment for solutions of varying viscosities by merely changing the frequency and power settings on the energy transducer. A further need exists for a system and method for cell sorting that is sufficiently economical to permit massively parallel, multi-channel sorting to obtain throughput and efficiency levels exceeding the capabilities of current instrumentation.

The general need clearly exists for increased separation flexibility by differentiating cells according to multiple parameters and multiple possible decisions depending upon quantification of the same parameter (non-binary decision making), e.g. differentiating cells into more than two groups based on any given parameter, without sacrificing cell purity or viability. An additional need exists in research for greater overall efficiency in sorting cells for end uses, e.g., in shortening total time between obtaining the cell mixture (for example a blood sample) and using the separated cells experimentally. In conventional separation systems efficiency is determined wholly by throughput, cell viability being equal, thus the tradeoff is between efficiency and purity for a given level of viability.

Often experimental uses require plating small numbers of a specific cell onto individual plates, dishes, wells, or arrays thereof, such as conventional well plates. Because all known cell sorting methods manipulate individual cells in a fluid to allow collection of a plurality of cells of a given sub-population, rather than permitting removal of an individual selected cell directly into a well plate well or other container, more steps are required between collecting the sample and experimentation. Considerable laboratory time and effort can be saved by direct delivery of a precisely known small number of cells from the sorted population into containers for use in experiments, rather than collecting the entire separated sub-population into a single container and subdividing the cells into experimental vessels. Non-binary methods which obtain greater enrichment than statistical enrichment methods can improve the overall efficiency purity tradeoff by reducing the number of steps required to effect a separation of several sub-populations, without increasing the of number of cells examined per second. Additionally delivering cells directly to an experimental receptacle or container, such as well of a well plate, can also improve the tradeoff between efficiency and purity without increasing throughput. Because both binary tagging and fractional enrichment methods manipulate the cells within the fluid rather than effecting ejection from the fluid entirely, high throughput, efficiency and purity while maintaining cell viability is limited. Thus a need exists for employing a means for the non-binary selective removal of viable cells from a mixture of cells directly into an experimental vessel. This can be effected by acoustic ejection.

No method or system is known to exist for sorting cells by ejecting individual cells from a fluid without killing the cells. Thus a need exists for a method and corresponding system for sorting cells by ejecting viable single cells from a fluid, preferably with non-binary selection and delivery of precise numbers of cells from a fluid directly into experimental containers. A method for ejecting single cells from a fluid is generally disclosed in copending application “Focused Acoustic Energy for Ejecting Cells in a Fluid” U.S. Ser. No. 09/727,391, inventors Mutz and Ellson, filed on Nov. 29, 2000, assigned to Picoliter, Inc. (Cupertino, Calif.), of which this application is a continuation in part. A method for cell sorting and system therefor based upon acoustic ejection of individual selected cells contained in droplets offers increased flexibility and overall efficiency without reduction of viability as compared to existing methods, by virtue of the ability to deliver sorted cells directly into experimental containers and sorting cells into several, rather than just two groups based on a single intrinsic or tagged property.

### SUMMARY OF THE INVENTION

Accordingly, it is an object of the present invention to provide systems and methods that overcome the above-mentioned disadvantages of the prior art.

In one aspect of the invention, a method is provided for acoustically ejecting from a container that is preferably a channel, a plurality of particles or localized, circumscribed volumes that can be single living cells contained in fluid droplets toward sites on a substrate surface or alternatively or in addition thereto into containers or channels sharing a plane common for deposition at a target array site or a container or channel by employing a device substantially as described in U.S. patent application Ser. No. 09/669,996 (“Acoustic Ejection of Fluids from a Plurality of Reservoirs”), inventors Ellson, Foote and Mutz, filed on Sep. 25, 2000, and assigned to Picoliter, Inc. (Cupertino, Calif.). As described in the aforementioned patent application, the ejecting device enables acoustic ejection of a plurality of fluid droplets toward designated sites on a substrate surface for deposition thereon, and: a plurality of cell containers or reservoirs each adapted to contain a fluid capable of carrying, for example, cells suspended therein; an acoustic ejector for generating acoustic radiation and a focusing means for focusing it at a focal point near the fluid surface in each of the reservoirs; and a means for positioning the ejector in acoustic coupling relationship to each of the cell containers or reservoirs. Preferably, each of the containers is removable, comprised of an individual well in a well plate, and/or arranged in an array. The cell containers or reservoirs, preferably channels, are preferably also substantially acoustically indistinguishable from one another, have appropriate acoustic impedance and attenuation to allow the energetically efficient focusing of acoustic energy near the surface of a contained fluid, and are capable of withstanding conditions of the fluid-containing reagent, here conditions permissive of cell viability.

In another aspect of the invention, a system is provided that is capable of selective sorting, into channels or other containers substantially transected by a common plane, parallel to a surface of the fluid, transecting the container from which cells are ejected by selective ejection of cells with adjustable velocity parallel to the fluid surface and simultaneously selectively forming an array of cells on a substrate surface comprising an array of substantially planar sites is provided, wherein each site contains a single cell. The operations of the system are performed by positioning an acoustic ejector so as to be in acoustically coupled relationship with a first carrier fluid cell suspension-containing reservoir containing a first carrier fluid and suspension of one cell type or clone, or a mixture of cell types or clones. After acoustic detection of the presence of a cell sufficiently close to the fluid surface, and detection of any properties used as criteria for ejection, by acoustic and/or electromagnetic wave measurements, the ejector is activated to generate and direct acoustic radiation so as to have a focal point within the carrier fluid and near the surface thereof, wherein the focal point contains a living cell, in an energy sufficient to eject a droplet of carrier fluid having a volume capable of containing a single cell, the droplet being ejected with a velocity vector having an component parallel to the plane of the fluid surface, thereby ejecting a single cell contained in fluid droplet toward a first target. Additional cells may be ejected from the first container. Or, the ejector may be repositioned so as to be in acoustically coupled relationship with a second carrier fluid cell suspension-containing reservoir, preferably a channel, and the process is repeated as above to eject a single cell contained in droplet of the a second fluid toward a second target, a coplanar container or fluidic channel or an array site on the substrate surface, wherein the first and second designated sites may or may not be the same. If desired, the method may be repeated with a plurality of cells from each container, with each reservoir generally although not necessarily containing a suspension of different cells or cell mixtures. The acoustic ejector is thus repeatedly repositioned so as to eject a single cell containing droplet from each reservoir toward a different designated site on a substrate surface. In such a way, the method is readily adapted for use in generating an array of cell on a substrate surface. The arrayed cells may be attached to the substrate surface by one or more specific binding systems each employing an external marker moiety that specifically recognizes a cognate moiety, such as a ligand receptor pair. An example of one such specific binding system being streptavidin as an external marker moiety, effected by transformation, with the cognate moiety being biotin. Multiple specific binding systems employing an external marker moiety displayed without cell transformation, include externally displayed Ig lymphocyte clones and epitopes as the cognate moiety.

Another aspect of the invention provides a method of forming arrays of single live cells more efficiently, rapidly, flexibly and economically than by other cell array approaches, while permitting efficient, continuous and simultaneous sorting of cells based upon selection by measurement of detectible properties quantitatively or semi-quantitatively, and multiple ejection targets selections permitting non-binary or severally branched decision making.

A further aspect of the invention is an integrated system and method for ejection of selected particles or circumscribed volumes, such as live cells, from a continuous stream of particles or circumscribed volumes flowing in fluidic ejection channels into flowing fluidic target channels based upon selection by measurement of detectible properties quantitatively or semi-quantitatively, and multiple ejection target selections permitting non-binary decision making integrated with the measurement by way of a processor.

Yet another aspect of the invention is an integrated system and method for ejection of live cells, from colonies of cells growing on a medium, typically agar or like semisolid or gel, onto a target substrate surface or into a target container or receptacle, such as a channel.

In yet a further aspect, the invention provides a method for facilitating acoustic ejection by spatially localized delivery of energy, preferably acoustic energy, to the region from which ejection is to occur prior to ejection.

### BRIEF DESCRIPTION OF THE DRAWINGS

FIGS. 1A and 1B, collectively referred to as FIG. 1, schematically illustrate in simplified cross-sectional view an embodiment of a device useful in conjunction with the invention, the device comprising first and second cell containers or reservoirs, an acoustic ejector, and an ejector positioning means. FIG. 1A shows the acoustic ejector acoustically coupled to the first cell container or reservoir and having been activated in order to eject a droplet of fluid containing a single cell from within the first cell container or reservoir toward a designated site on a substrate surface. FIG. 1B shows the acoustic ejector acoustically coupled to a second cell container or reservoir.

FIGS. 2A, 2B and 2C, collectively referred to as FIG. 2, illustrate in schematic view a variation of the inventive embodiment of FIG. 1 wherein the cell containers or reservoirs comprise individual wells in a reservoir well plate and the substrate comprises a smaller well plate with a corresponding number of wells. FIG. 2A is a schematic top plane view of the two well plates, i.e., the cell container or reservoir well plate and the substrate surface having arrayed cells contained in fluid droplets. FIG. 2B illustrates in cross-sectional view a device comprising the cell container or reservoir well plate of FIG. 2A acoustically coupled to an acoustic ejector, wherein a cell contained in a droplet is ejected from a first well of the cell container or reservoir well plate into a first well of the substrate well plate. FIG. 2C illustrates in cross-sectional view the device illustrated in FIG. 2B, wherein the acoustic ejector is acoustically coupled to a second well of the cell container or reservoir well plate and further wherein the device is aligned to enable the acoustic ejector to eject a droplet from the second well of the cell container or reservoir well plate to a second well of the substrate well plate.

FIGS. 3A, 3B, 3C and 3D, collectively referred to as FIG. 3, schematically illustrate in simplified cross-sectional view an embodiment of the inventive method in which cells having an externally displayed marker moiety are ejected onto a substrate using the device of FIG. 1. FIG. 3A illustrates the ejection of a cell containing fluid droplet onto a designated site of a substrate surface. FIG. 3B illustrates the ejection of a droplet containing a first cell displaying a first marker moiety adapted for attachment to a modified substrate surface to which a first cognate moiety is attached. FIG. 3C illustrates the ejection of a droplet of second fluid containing a second cell displaying a second molecular moiety adapted for attachment to the a different site on the surface. FIG. 3D illustrates the substrate and the first and second cells arrayed thereon by the process illustrated in FIGS. 3A, 3B and 3C.

FIGS. 4A and 4B, collectively referred to as FIG. 4, depict arrayed cells contained in droplets deposited by acoustic ejection using the device of FIG. 1. FIG. 4A illustrates two different cells resident at adjacent array sites, contained in fluid droplets adhering to a designated site of a substrate surface by surface tension, with each cell further attached to the site by binding of streptavidin (SA) to a biotinylated (biotin (B) linked) surface. Streptavidin is displayed on the cell exterior as a result of transformation by an external display targeted streptavidin coding sequence containing construct. FIG. 4B illustrates two different cells resident at adjacent array sites, contained in fluid droplets adhering to a designated site of a substrate surface by surface tension, with each cell further attached to the site by binding of an externally displayed antigenic epitope characteristic to the cell (here E1 and E2) to a two different monoclonal antibodies (mAb-E1, mAb-E2) specific respectively for the different epitopes, each mAb linked to the surface at only one of the adjacent array sites.

FIGS. 5A, 5B, 5C, 5D and 5E, collectively referred to as FIG. 5, depict a device having a fluidic channel as the container from which the cells are ejected onto the substrate. FIG. 5A and FIG. 5B illustrate the device as a schematic. 5C illustrates top view of channels containing live cells the substrate surface having arrayed cells contained in fluid droplets. FIG. 5D illustrates a cross section of a channel showing a physical upwards protrusion of channel floor to direct cells to be sufficiently close to fluid surface for ejection. FIG. 5E illustrates a cross section of a channel showing use of focused energy, such as acoustic energy, to direct cells to be sufficiently close to fluid surface for ejection.

FIG. 6 depicts a top view of a central channel, an ejection channel, with two detecting devices D₁ and D₂ past which cells flow and two ejection sites, represented by large ellipses, each containing a depiction of a cell, from which cells may be ejected perpendicular to the surface onto a substrate (not shown), or into adjacent target channels.

FIGS. 7A and 7B, collectively referred to as FIG. 7, depict a device having a central fluidic channel that feeds cells with high throughput laterally to a peripheral channel from which the cells are ejected onto the substrate, preferably by use of multiple ejectors. FIG. 7A illustrates a side view of a vertical channel containing cells within a larger vessel. The periphery of the larger vessel is fluidically accessible from the vertical channel only by passing under an angled lip projecting laterally from the vertical channel with the distance between the lip and the floor of the larger vessel decreasing radially outward so that cells can pass radially outwards from the central channel, to the periphery. At the periphery a channel is formed where cells are spaced further apart, relative to spacing in the vertical channel. FIG. 7B, top view, showing cells along the side walls of the larger vessel allowing simultaneous ejection of a large number of cells by use of multiple ejectors to effect a high throughput and efficiency.

### DETAILED DESCRIPTION OF THE INVENTION

Before describing the present invention in detail, it is to be understood that this invention is not limited to specific fluids, cells, biomolecules or device structures, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a cell container” or “a reservoir” includes a plurality of cell containers or reservoirs, reference to a fluid” includes a plurality of fluids, reference to “a biomolecule” includes a combination of biomolecules, and the like.

In describing and claiming the present invention, the following terminology will be used in accordance with the definitions set out below.

The terms “acoustic coupling” and “acoustically coupled” used herein refer to a state wherein an object is placed in direct or indirect contact with another object so as to allow acoustic radiation to be transferred between the objects without substantial loss of acoustic energy. When two items are indirectly acoustically coupled, an “acoustic coupling medium” is needed to provide an intermediary through which acoustic radiation may be transmitted. Thus, an ejector may be acoustically coupled to a fluid, e.g., by immersing the ejector in the fluid or by interposing an acoustic coupling medium between the ejector and the fluid to transfer acoustic radiation generated by the ejector through the acoustic coupling medium and into the fluid.

The term “adsorb” as used herein refers to the noncovalent retention of a molecule by a substrate surface. That is, adsorption occurs as a result of noncovalent interaction between a substrate surface and adsorbing moieties present on the molecule that is adsorbed. Adsorption may occur through hydrogen bonding, van der Waal's forces, polar attraction or electrostatic forces (i.e., through ionic bonding). Examples of adsorbing moieties include, but are not limited to, amine groups, carboxylic acid moieties, hydroxyl groups, nitroso groups, sulfones and the like. Often the substrate may be functionalized with adsorbent moieties to interact in a certain manner, as when the surface is functionalized with amino groups to render it positively charged in a pH neutral aqueous environment. Likewise, adsorbate moieties may be added in some cases to effect adsorption, as when a basic protein is fused with an acidic peptide sequence to render adsorbate moieties that can interact electrostatically with a positively charged adsorbent moiety.

The term “array” used herein refers to a two-dimensional arrangement of features such as an arrangement of reservoirs (e.g., wells in a well plate) or an arrangement of different materials including ionic, metallic or covalent crystalline, including molecular crystalline, composite or ceramic, glassine, amorphous, fluidic or molecular materials on a substrate surface (as in an oligonucleotide or peptidic array). Different materials in the context of molecular materials includes chemical isomers, including constitutional, geometric and stereoisomers, and in the context of polymeric molecules constitutional isomers having different monomer sequences. Arrays are generally comprised of regular, ordered features, as in, for example, a rectilinear grid, parallel stripes, spirals, and the like, but non-ordered arrays also may be used. An array is distinguished from the more general term pattern in that patterns do not necessarily contain regular and ordered features. The arrays or patterns formed using the devices and methods of the invention have no optical significance to the unaided human eye. For example, the invention does not involve ink printing on paper or other substrates in order to form letters, numbers, bar codes, figures, or other inscriptions that have visual significance to the unaided human eye. In addition, arrays and patterns formed by the deposition of ejected droplets on a surface as provided herein are preferably substantially invisible to the unaided human eye. Arrays typically but do not necessarily comprise at least about 4 to about 10,000,000 features, generally in the range of about 4 to about 1,000,000 features.

The term “attached,” as in, for example, a substrate surface having a molecular moiety “attached” thereto (e.g., in the individual molecular moieties in arrays generated using the methodology of the invention) includes covalent binding, adsorption, and physical immobilization. The terms “binding” and “bound” are identical in meaning to the term “attached.”

The term “binary” refers to a two possibility selection scheme, for example ejection or non-ejection based upon the detection of a threshold level of fluorescence. The term “non-binary” refers to selection schemes having more than two possible selections, for example ejection to a first target container based upon a detection of a fluorescence emission greater than a high threshold, ejection to a second target container based upon fluorescence detected above the detection threshold, but below the high fluorescence threshold, or non-ejection if no fluorescence of a given frequency is detectable.

The term “biomolecule” as used herein refers to any organic molecule, whether naturally occurring, recombinantly produced, or chemically synthesized in whole or in part, that is, was or can be a part of a living organism, or synthetic analogs of molecules occurring in living organisms including nucleic acid analogs having peptide backbones and purine and pyrimidine sequence, carbamate backbones having side chain sequence resembling peptide sequences, and analogs of biological molecules such as epinephrine, GABA, endorphins, interleukins and steroids. The term encompasses, for example, nucleotides, amino acids and monosaccharides, as well as oligomeric and polymeric species such as oligonucleotides and polynucleotides, peptidic molecules such as oligopeptides, polypeptides and proteins, saccharides such as disaccharides, oligosaccharides, polysaccharides, mucopolysaccharides or peptidoglycans (peptido-polysaccharides) and the like. The term also encompasses synthetic GABA analogs such as benzodiazepines, synthetic epinephrine analogs such as isoproterenol and albuterol, synthetic glucocorticoids such as prednisone and betamethasone, and synthetic combinations of naturally occurring biomolecules with synthetic biomolecules, such as theophylline covalently linked to betamethasone.

The term “colony of cells” or “cell colony” as used herein refers to one or more cells. In the case that a plurality of cells comprise the colony, the cells are sufficiently close that the environment or external conditions of a given single cell is affected by neighboring cells.

It will be appreciated that, as used herein, the terms “nucleoside” and “nucleotide” refer to nucleosides and nucleotides containing not only the conventional purine and pyrimidine bases, i.e., adenine (A), thymine (T), cytosine (C), guanine (G) and uracil (U), but also protected forms thereof, e.g., wherein the base is protected with a protecting group such as acetyl, difluoroacetyl, trifluoroacetyl, isobutyryl or benzoyl, and purine and pyrimidine analogs. Suitable analogs will be known to those skilled in the art and are described in the pertinent texts and literature. Common analogs include, but are not limited to, 1-methyladenine, 2-methyladenine, N⁶-methyladenine, N⁶-isopentyladenine, 2-methylthio-N⁶-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “oligonucleotide” shall be generic to polydeoxynucleotides (containing 2-deoxy-D-ribose), to polyribonucleotides (containing D-ribose), to any other type of polynucleotide which is an N-glycoside of a purine or pyrimidine base, and to other polymers containing nonnucleotidic backbones (for example PNAs), providing that the polymers contain nucleobases in a configuration that allows for base pairing and base stacking, such as is found in DNA and RNA. Thus, these terms include known types of oligonucleotide modifications, for example, substitution of one or more of the naturally occurring nucleotides with an analog, inter-nucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.).

There is no intended distinction in length between the term “polynucleotide” and “oligonucleotide,” and these terms will be used interchangeably. These terms refer only to the primary structure of the molecule. As used herein the symbols for nucleotides and polynucleotides are according to the IUPAC-IUB Commission of Biochemical Nomenclature recommendations (Biochemistry 9:4022, 1970).

“Peptidic” molecules refer to peptides, peptide fragments, and proteins, i.e., oligomers or polymers wherein the constituent monomers are alpha amino acids linked through amide bonds. The amino acids of the peptidic molecules herein include the twenty conventional amino acids, stereoisomers (e.g., D-amino acids) of the conventional amino acids, unnatural amino acids such as α,α-disubstituted amino acids, N-alkyl amino acids, and other unconventional amino acids. Examples of unconventional amino acids include, but are not limited to, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine.

The term “fluid” as used herein refers to matter that is nonsolid or at least partially gaseous and/or liquid. A fluid may contain a solid that is minimally, partially or fully solvated, dispersed or suspended; particles comprised of gels or discrete fluids may also be suspended in a fluid. Examples of fluids include, without limitation, aqueous liquids (including water per se and salt water) and nonaqueous liquids such as organic solvents and the like. Live cells suspended in a carrier fluid is an example of a gel or discrete fluid suspended in a fluid. As used herein, the term “fluid” is not synonymous with the term “ink” in that an ink must contain a colorant and may not be gaseous and/or liquid.

The phrase “localized fluid volume” refers to a spatially localized volume of fluid. Typically a localized fluid volume will have different physical properties than the surrounding fluid, although this is not required by the definition. In practice a localized fluid volume can only be detected if its properties are different from the surrounding fluid. A sugar crystal suspended in an unsaturated (by the sugar) aqueous solution, and surrounded by a volume in which the sugar concentration of the local fluid is greater than the mean sugar concentration of the bulk fluid is an example of an uncircumscribed localized fluid volume having no delineating or circumscribing structure. A “circumscribed fluid volume” is a localized fluid volume which is delineated or circumscribed, usually by a structure, but possibly also by a potential well of an energetic field. A biological cell is a prime example of a circumscribed fluid volume, as it is delineated by the cell membrane structure. Other examples of circumscribed fluid volumes include platelets, mitochondria and nuclei, which are cell organelles or packaged cellular subdivisions. An example of a circumscribed volume not derived from a living organism is a fluid containing microcapsule. The fluid in a circumscribed fluid volume may contain suspended solid and gel particles. But by being circumscribed the entire circumscribed volume behaves as a single particle unless the circumscribing structure or field is breached. A solid or gel particle,such as a glass or polymer bead, is included within the contemplated meaning of circumscribed volume, being circumscribed from the carrier fluid by the nature of the material from which it is made.

The term “acoustic focusing means” as used herein refers to causing acoustic waves to converge at a focal point by either a device separate from the acoustic energy source that acts like an optical lens, or by the spatial arrangement of acoustic energy sources to effect convergence of acoustic energy at a focal point by constructive and destructive interference, as by use of a phased array of acoustic sources to effect constructive interference. A focusing means may be as simple as a solid member having a curved surface, or it may include complex structures such as those found in Fresnel lenses, which employ diffraction in order to direct acoustic radiation.

The term “reservoir” as used herein refers a receptacle or chamber for holding or containing a fluid. Thus, a fluid in a reservoir necessarily has a free surface, i.e., a surface that allows a droplet to be ejected therefrom. As long as a fluid container has at least one free surface from which fluid can be ejected, the container is a reservoir regardless of specific geometry. Thus reservoir contemplates, for example, a microfluidic channel having flowing fluid from which droplets are ejected, a contained particle plasma, and a fluid sphere, held together by inter-atomic or intermolecular forces, floating in a zero-gravity environment. A “cell container” or “cell reservoir” is a reservoir which is specialized for ejection of living cells suspended in a carrier fluid, and includes, by example a microfluidic or other channel through which living cells flow suspended in a carrier fluid.

The term “substrate” as used herein refers to any material having a surface onto which one or more cells contained in a droplet of carrier fluid may be deposited. The substrate may be constructed in any of a number of forms such as wafers, slides, well plates, membranes, for example. In addition, the substrate may be porous or nonporous as may be required for any particular fluid deposition. Suitable substrate materials include, but are not limited to, supports that are typically used for solid phase chemical synthesis and/or biological containers or reservoirs as those used for tissue or cell culture, including polymeric materials (e.g., polydimethylsiloxane, polyethylene glycol (PEG), polystyrene, polyvinyl acetate, polyvinyl chloride, polyvinyl pyrrolidone, polyacrylonitrile, polyacrylamide, polymethyl methacrylate, polytetrafluoroethylene, polyethylene, polypropylene, polyvinylidene fluoride, polycarbonate, divinylbenzene styrene-based polymers), agarose (e.g., Sepharose®), dextran (e.g., Sephadex ®), cellulosic polymers and other polysaccharides, silica and silica-based materials, glass (particularly controlled pore glass, or “CPG”) and functionalized glasses, ceramics, and such substrates treated with surface coatings, e.g., with microporous polymers (particularly cellulosic polymers such as nitrocellulose and spun synthetic polymers such as spun polyethylene), metallic compounds (particularly microporous aluminum), or the like. While the foregoing support materials are representative of conventionally used substrates, it is to be understood that the substrate may in fact comprise any biological, nonbiological, organic and/or inorganic material, and may be in any of a variety of physical forms, e.g., particles, strands, precipitates, gels, sheets, tubing, spheres, containers, capillaries, pads, slices, films, plates, slides, and the like, and may further have any desired shape, such as a disc, square, sphere, circle, etc. The substrate surface may or may not be flat, e.g., the surface may contain raised or depressed regions.

A substrate may additionally contain or may be derivatized to contain reactive functionality which covalently links a compound to the surface thereof. These are widely known and include, for example, silicon dioxide supports containing reactive Si—OH groups, polyacrylamide supports, polystyrene supports, polyethylene glycol supports, and the like. Alternatively a moiety which binds to a cognate moiety, for example a ligand receptor pair may be employed to specifically attach a molecule, particle, living cell, biological tissue or tissue component or the like to a substrate surface. One example of attachment using a cognate moiety pair employs a surface that is covalently linked to the ligand biotin, a type of biotin functionalized or biotinylated surface, and the receptor protein streptavidin which specifically binds biotin in a reversible non-covalent manner typical of ligand receptor interactions. Macromolecules such as fusion proteins comprising streptavidin, solid or gel particles to which streptavidin is securely attached and cells transformed to externally display streptavidin may be attached to the biotinylated substrate surface.

The term “surface modification” as used herein refers to the chemical and/or physical alteration of a surface by an additive or subtractive process to change one or more chemical and/or physical properties of a substrate surface or a selected site or region of a substrate surface. For example, surface modification may involve (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface. Thus an example of a surface modification by functionalization is the biotinylated surface that can be used in conjunction with the receptor streptavidin to effect various attachments. The grafting of polymers such as PEG onto surfaces of materials such as Si is another example of surface functionalization.

In one embodiment, then, the invention pertains to a device for acoustically ejecting a plurality of single cell containing droplets toward one or more designated sites on a substrate surface. The device comprises one or more cell containers or reservoirs, each adapted to contain a carrier fluid within which living cells are suspended; an ejector comprising an acoustic radiation generator for generating acoustic radiation and a focusing means for focusing acoustic radiation at a focal point within and near the fluid surface in each of the reservoirs; and a means for positioning the ejector in acoustic coupling relationship to each of the reservoirs.

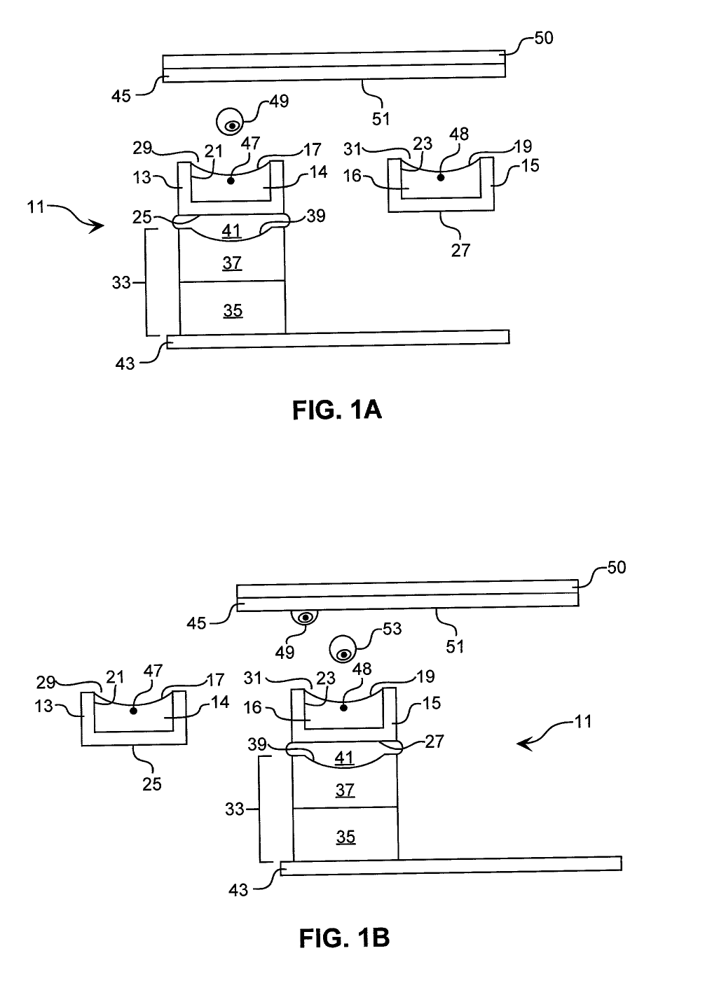

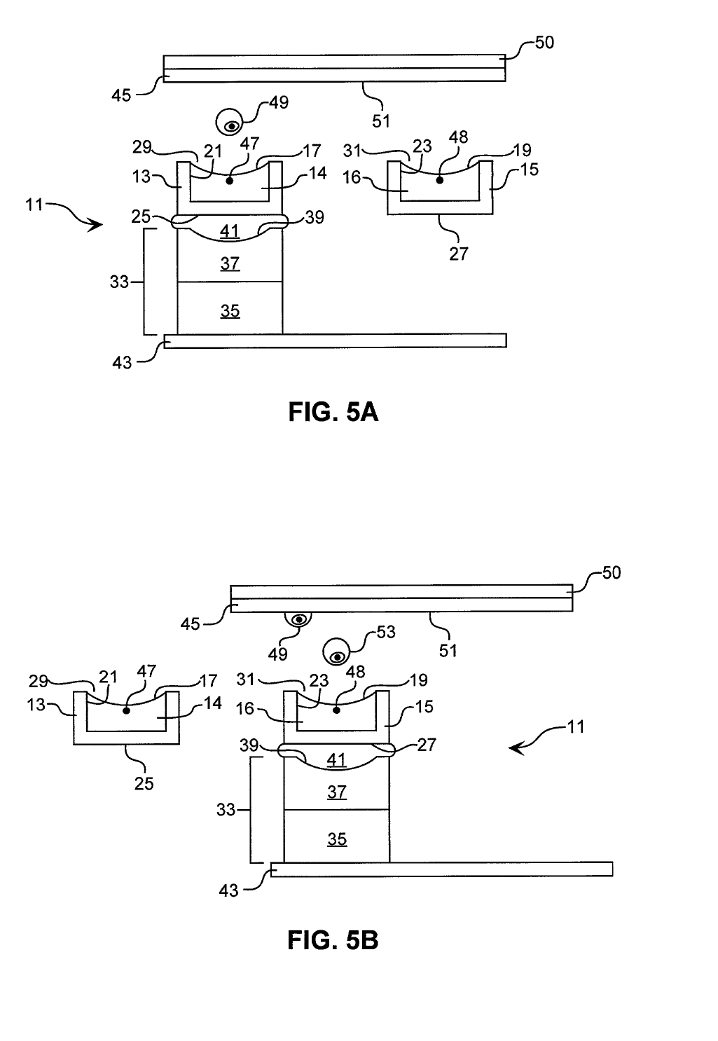

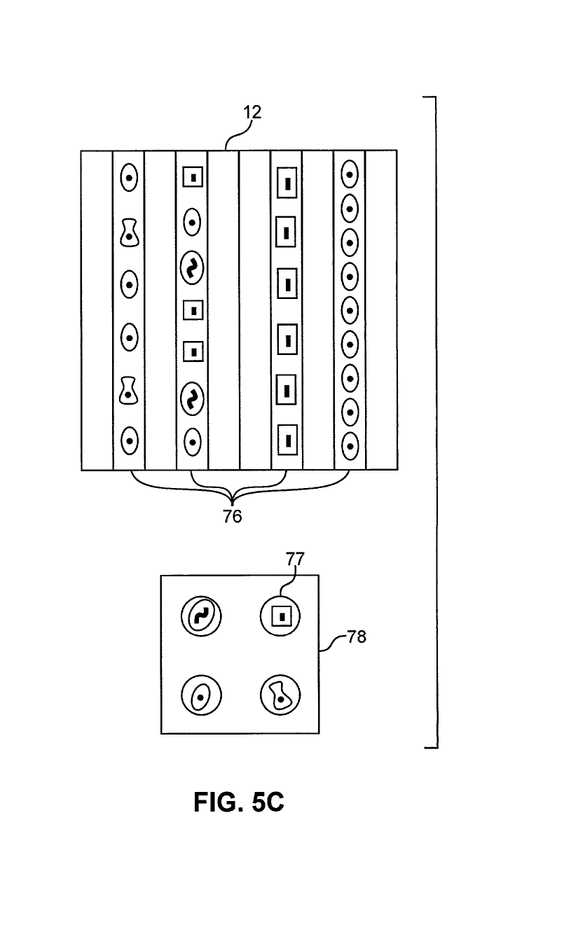

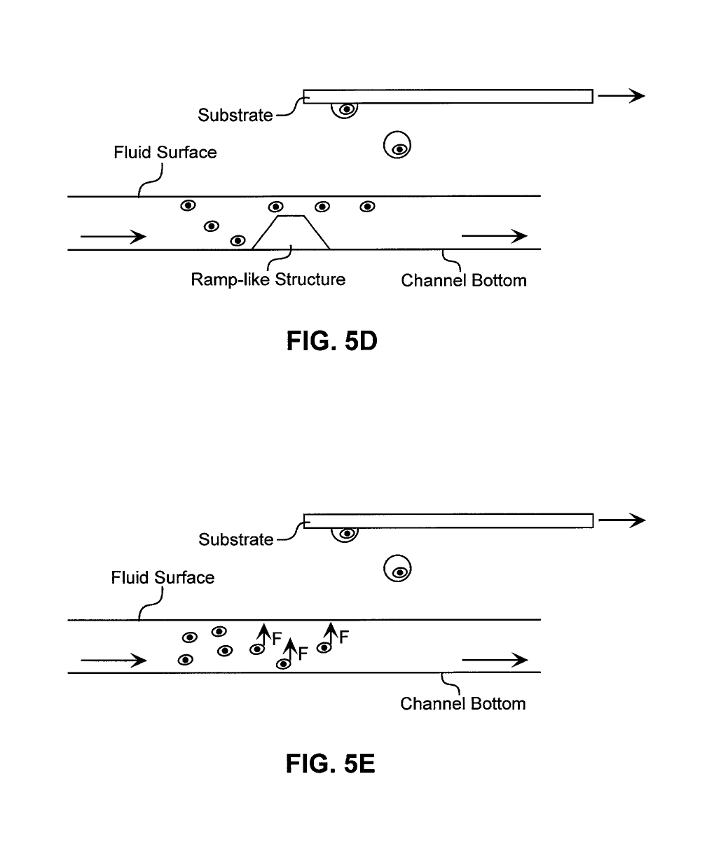

FIGS. 1 and 5 illustrate alternative embodiments of the employed device in simplified cross-sectional view. FIG. 1 depicts a cell ejection system where the cell container or reservoir is a conventional container, such as a conventional petri dish, which is radially symmetric. In FIG. 5, the cell reservoir is a fluidic channel, through which live cells flow in a carrier fluid. As with all figures referenced herein, in which like parts are referenced by like numerals, FIGS. 1 and 5 are not to scale, and certain dimensions may be exaggerated for clarity of presentation. The device 11 includes a plurality of cell containers or reservoirs, i.e., at least two containers or reservoirs, with a first cell container indicated at 13 and a second container indicated at 15, each adapted to contain a fluid, in which live cells are suspended, having a fluid surface, e.g., a first cell container having cells suspended in fluid 14 and a second cell container having cells suspended in fluid 16 having fluid surfaces respectively indicated at 17 and 19. The suspended cells and carrier fluids of 14 and 16 may be the same or different. As depicted, the cell containers or reservoirs are of substantially identical construction so as to be substantially acoustically indistinguishable, but identical construction is not a requirement. The cell containers are shown as separate removable components but may, if desired, be fixed within a plate or other substrate. For example, the plurality of containers in FIG. 1 may comprise individual wells in a well plate, optimally although not necessarily arranged in an array. Likewise, the plurality of containers in FIG. 5 may comprise separate channels or individual channels in a plate, by example a pattern of individual microfluidic channels etched into a plate as by photolithography. Each of the cell containers or reservoirs 13 and 15 is preferably bilaterally (FIG. 5—channels) or axially (FIG. 1) symmetric, having substantially vertical walls 21 and 23 extending upward from reservoir bases 25 and 27 and terminating at openings 29 and 31, respectively, although other reservoir shapes may be used, including enclosed fluidic channels having an aperture or opening for ejection at a specific location. The material and thickness of each cell container or reservoir base should be such that acoustic radiation may be transmitted therethrough and into the fluid contained within the reservoirs.

The device embodiments depicted in FIGS. 1 and 5 also include an acoustic ejector 33 comprised of an acoustic radiation generator 35 for generating acoustic radiation and a focusing means 37 for focusing the acoustic radiation at a focal point within the fluid from which a droplet is to be ejected, near the fluid surface. As shown in FIGS. 1 and 5, the focusing means 37 may comprise a single solid piece having a concave surface 39 for focusing acoustic radiation, but the focusing means may be constructed in other ways as discussed below. The acoustic ejector 33 is thus adapted to generate and focus acoustic radiation so as to eject a droplet of fluid from each of the fluid surfaces 17 and 19 when acoustically coupled to reservoirs 13 and 15 and thus to fluids 14 and 16, respectively. The acoustic radiation generator 35 and the focusing means 37 may function as a single unit controlled by a single controller, or they may be independently controlled, depending on the desired performance of the device. Typically, single ejector designs are preferred over multiple ejector designs because accuracy of droplet placement and consistency in droplet size and velocity are more easily achieved with a single ejector.

As will be appreciated by those skilled in the art, any of a variety of focusing means may be employed in conjunction with the present invention. For example, one or more curved surfaces may be used to direct acoustic radiation to a focal point near a fluid surface. One such technique is described in U.S. Pat. No. 4,308,547 to Lovelady et al. Focusing means with a curved surface have been incorporated into commercially available acoustic transducers such as those manufactured by Panametrics Inc. (Waltham, Mass.). In addition, Fresnel lenses are known in the art for directing acoustic energy at a predetermined focal distance from an object plane. See, e.g., U.S. Pat. No. 5,041,849 to Quate et al. Fresnel lenses may have a radial phase profile that diffracts a substantial portion of acoustic energy into a predetermined diffraction order at diffraction angles that vary radially with respect to the lens. The diffraction angles should be selected to focus the acoustic energy within the diffraction order on a desired object plane. Phased arrays of acoustic energy emitters have also been used to focus acoustic energy at a specified point as a result of constructive and destructive interference between the acoustic waves emitted by the arrayed sources (Amemiya et al (1997) Proceeding of 1997 IS&T NIP13 International Conference on Digital Printing Technologies Proceedings, pp. 698-702.).

There are also a number of ways to acoustically couple the ejector 33 to each individual reservoir and thus to the fluid therein. One such approach is through direct contact as is described, for example, in U.S. Pat. No. 4,308,547 to Lovelady et al., wherein a focusing means constructed from a hemispherical crystal having segmented electrodes is submerged in a liquid to be ejected. The aforementioned patent further discloses that the focusing means may be positioned at or below the surface of the liquid. However, this approach for acoustically coupling the focusing means to a fluid is undesirable when the ejector is used to eject different fluids in a plurality of containers or reservoirs, as repeated cleaning of the focusing means would be required in order to avoid cross-contamination. The cleaning process would necessarily lengthen the transition time between each droplet ejection event. In addition, in such a method, cells in the fluid would adhere to the ejector as it is removed from a container, wasting cellular material that may be rare or irreplaceable. Finally, submersion in the fluid is not possible with conventional acoustic energy focusing means when the reservoirs are microfabricated, as when the cell containers are microfluidic channels or micro-wells, because the containers are too small.

One of skill in the art of microfabrication would be able to make a focusing means comprising a microfabricated curved member. Similarly a microfabricated focusing means constructed from a hemispherical crystal having segmented electrodes, e.g. a miniature focusing means as described in U.S. Pat. No. 4,308,547 to Lovelady et al., can be made by routine microfabrication techniques. Submersion would then be possible with the same disadvantages as above. For microfluidic channels or wells, then, a focusing means as well as a source of acoustic energy could be integrated into the microfabricated assembly.

An approach practicable for any reservoir dimensions would be to acoustically couple a conventional non-microfabricated or macro-scale ejector to the reservoirs and reservoir fluids without contacting any portion of the ejector, e.g., the focusing means, with any of the fluids to be ejected. To this end, the present invention provides an ejector positioning means for positioning the ejector in controlled and repeatable acoustic coupling with each of the fluids in the cell containers or reservoirs to eject droplets therefrom without submerging the ejector therein. This typically involves direct or indirect contact between the ejector and the external surface of each reservoir. When direct contact is used in order to acoustically couple the ejector to each reservoir, it is preferred that the direct contact is wholly conformal to ensure efficient acoustic energy transfer. That is, the ejector and the reservoir should have corresponding surfaces adapted for mating contact. Thus, if acoustic coupling is achieved between the ejector and reservoir through the focusing means, it is desirable for the reservoir to have an outside surface that corresponds to the surface profile of the focusing means. Without conformal contact, efficiency and accuracy of acoustic energy transfer may be compromised. In addition, since many focusing means have a curved surface, the direct contact approach may necessitate the use of reservoirs having a specially formed inverse surface.

Optimally, acoustic coupling is achieved between the ejector and each of the reservoirs through indirect contact, as illustrated in FIGS. 1A and 5A. In the figure, an acoustic coupling medium 41 is placed between the ejector 33 and the base 25 of reservoir 13, with the ejector and reservoir located at a predetermined distance from each other. The acoustic coupling medium may be an acoustic coupling fluid, preferably an acoustically homogeneous material in conformal contact with both the acoustic focusing means 37 and each reservoir. In addition, it is important to ensure that the fluid medium is substantially free of material having different acoustic properties than the fluid medium itself. As shown, the first reservoir 13 is acoustically coupled to the acoustic focusing means 37 such that an acoustic wave is generated by the acoustic radiation generator and directed by the focusing means 37 into the acoustic coupling medium 41, which then transmits the acoustic radiation into the reservoir 13.

In operation, reservoirs 13 and 15 of the device are each filled with first and second carrier fluids having cells or cell mixtures suspended therein 14 and 16, respectively, as shown in FIGS. 1 and 5. The acoustic ejector 33 is positionable by means of ejector positioning means 43, shown below reservoir 13, in order to achieve acoustic coupling between the ejector and the reservoir through acoustic coupling medium 41. Substrate 45 is positioned above and in proximity to the first reservoir 13 such that one surface of the substrate, shown in FIGS. 1 and 5 as underside surface 51, faces the reservoir and is substantially parallel to the surface 17 of the fluid 14 therein. Once the ejector, the reservoir and the substrate are in proper alignment, the acoustic radiation generator 35 is activated to produce acoustic radiation that is directed by the focusing means 37 to a focal point 47 near the fluid surface 17 of the first reservoir. As a result, droplet 49 is ejected from the fluid surface 17 onto a designated site on the underside surface 51 of the substrate. The ejected droplet may be retained on the substrate surface by solidifying thereon after contact; in such an embodiment, it is necessary to maintain the substrate at a low temperature, i.e., a temperature that results in droplet solidification after contact. Alternatively, or in addition, a molecular moiety or marker moiety displayed on the surface of the droplet contained cell attaches to the substrate surface after contract, through adsorption, physical immobilization, or covalent binding.

Then, as shown in FIGS. 1B and 5B, a substrate positioning means 50 repositions the substrate 45 over reservoir 15 in order to receive a droplet therefrom at a second designated site. FIGS. 1B and 5B also show that the ejector 33 has been repositioned by the ejector positioning means 43 below reservoir 15 and in acoustically coupled relationship thereto by virtue of acoustic coupling medium 41. Once properly aligned as shown in FIGS. 1B and 5B, the acoustic radiation generator 35 of ejector 33 is activated to produce acoustic radiation that is then directed by focusing means 37 to a focal point within fluid 16 near the fluid surface 19, thereby ejecting droplet 53 onto the substrate. It should be evident that such operation is illustrative of how the employed device may be used to eject a plurality of single cells contained in fluid droplets from reservoirs in order to form a pattern, e.g., an array, of cells on the substrate surface 51. It should be similarly evident that the device may be adapted to eject a plurality of individual cells contained in ejected fluid droplets from one or more reservoirs onto the same site of the substrate surface.

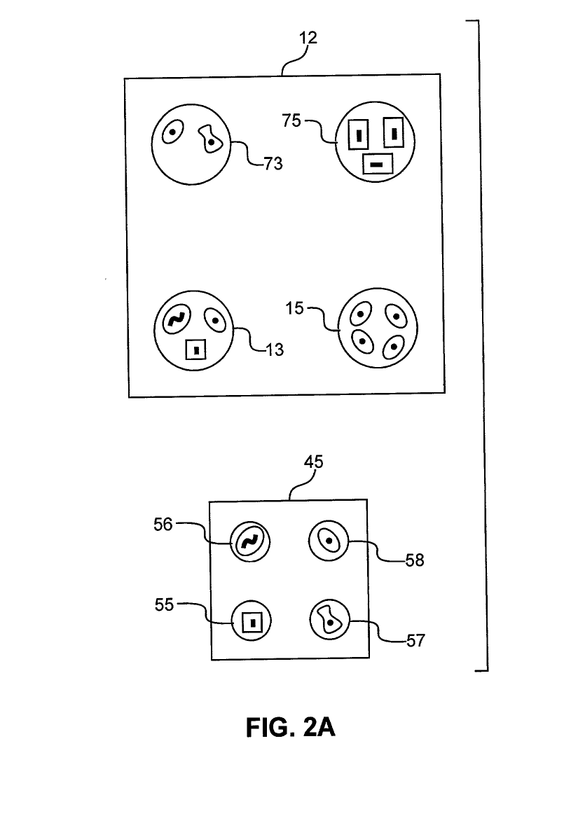

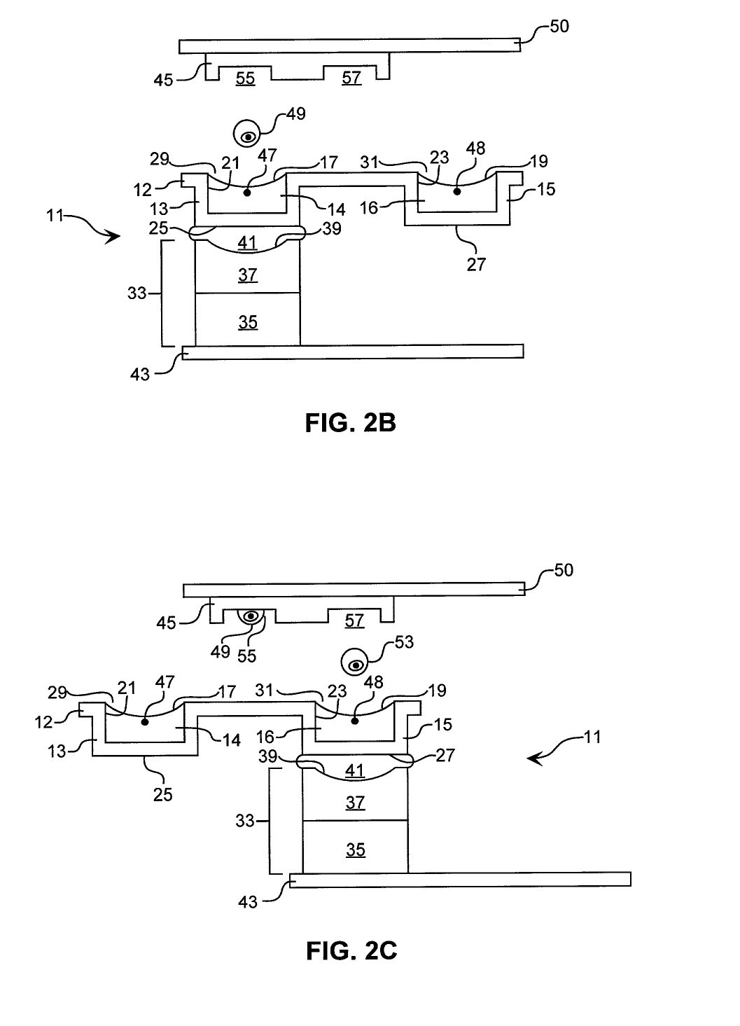

In another embodiment, the device is constructed so as to allow transfer of cells contained in fluid droplets between well plates, in which case the substrate comprises a substrate well plate, and the fluid suspended cell-containing reservoirs are individual wells in a reservoir well plate. FIG. 2 illustrates such a device, wherein four individual wells 13, 15, 73 and 75 in reservoir well plate 12 serve as fluid reservoirs for containing a plurality of a specific type of cell or a mixture of different cell types suspended in a fluid for ejection of droplets containing a single cell, and the substrate comprises a smaller well plate 45 of four individual wells indicated at 55, 56, 57 and 58. FIG. 2A illustrates the cell container or reservoir well plate and the substrate well plate in top plane view. As shown, each of the well plates contains four wells arranged in a two-by-two array. FIG. 2B illustrates the employed device wherein the cell container or reservoir well plate and the substrate well plate are shown in cross-sectional view along wells 13, 15 and 55, 57, respectively. As in FIGS. 1 and 5, reservoir wells 13 and 15 respectively contain cells suspended in carrier fluids 14 and 16 having carrier fluid surfaces respectively indicated at 17 and 19. The materials and design of the wells of the cell container or reservoir well plate are similar to those of the containers illustrated in FIGS. 1 and 5. For example, the cell containers or reservoirs shown in FIG. 2B (wells) and in FIG. 5B (channels) are of substantially identical construction so as to be substantially acoustically indistinguishable. In these embodiments, the bases of the cell reservoirs are of a material (e.g. a material having appropriate acoustic impedance) and thickness so as to allow efficient transmission of acoustic radiation therethrough into the contained carrier fluid.

The device of FIGS. 2 and 5 also includes an acoustic ejector 33 having a construction similar to that of the ejector illustrated in FIG. 1, comprising an acoustic generating means 35 and a focusing means 37. FIG. 2B shows the ejector acoustically coupled to a reservoir well through indirect contact; that is, an acoustic coupling medium 41 is placed between the ejector 33 and the reservoir well plate 12, i.e., between the curved surface 39 of the acoustic focusing means 37 and the base 25 of the first cell container or reservoir (well or channel) 13. As shown, the first cell container or reservoir (well or channel) 13 is acoustically coupled to the acoustic focusing means 37 such that acoustic radiation generated in a generally upward direction is directed by the focusing means 37 into the acoustic coupling medium 41, which then transmits the acoustic radiation into the cell container or reservoir (well or channel) 13.

In operation, each of the cell containers or reservoirs (well or channel) is preferably filled with a carrier fluid having a different type of cell or mixture of cells suspended within the carrier fluid. As shown, reservoir wells 13 and 15 of the device are each filled with a carrier fluid having a first cell mixture 14 and a carrier fluid having a second cell mixture 16, as in FIG. 1, to form fluid surfaces 17 and 19, respectively. FIGS. 1 and 5 show that the ejector 33 is positioned below reservoir well 13 by an ejector positioning means 43 in order to achieve acoustic coupling therewith through acoustic coupling medium 41.

For the ejection of individual cells into well plates from cell containers, FIG. 2A, the first substrate well 55 of substrate well plate 45 is positioned above the first reservoir well 13 in order to receive a droplet ejected from the first cell container or reservoir (well or channel).

Once the ejector, the cell container or reservoir (well or channel) and the substrate are in proper alignment, the acoustic radiation generator is activated to produce an acoustic wave that is focused by the focusing means to direct the acoustic wave to a focal point 47 near fluid surface 17, with the amount of energy being insufficient to eject fluid. This first emission of focused acoustic energy permits sonic detection of the presence of a cell sufficiently close to the surface for ejection by virtue of reflection of acoustic energy created by a difference in acoustic impedance between the cell and carrier fluid. Methods for determining the position of the cell by sonic detection are readily apprehended by those of ordinary skill in the art of acoustic microscopy and related arts. After a cell is detected and localized, other properties may be measured before the decision to eject is made. Also, if no cell is sufficiently close to the surface for ejection, the acoustic energy may be focused at progressively greater distances from the fluid surface until a cell is located and driven closer to the surface by focused acoustic energy or other means. For example, a uniform field such as a photon field which will exert a force based on cross sectional area and change in photon momentum, determined by the difference of refractive indices of the carrier medium and the cells. An electric field that exerts a force based on net surface charge can be used to move cells. A carrier fluid having a low density relative to the cells or a carrier fluid comprising a density gradient can also be used to position cells as for ejection. It will be appreciated that numerous ways of effecting a short mean cell distance from the fluid surface exists. For channels, especially microfabricated channels, mechanical or fluidic means may be used to effect a sufficiently small distance from the fluid surface by placing a ramp like structure across the channel that decreases channel depth over the ramp to a depth on the order of the cell diameter, thereby only permitting cells to flow near the surface; cells are unlikely to jam at the ramp because the fluid velocity will be highest where the channel depth is lowest as depicted in FIG. 5D. FIG. 5E depicts a microfluidic channel where a force acting on the cells moves them towards the surface.

Because microfluidic channels may be fabricated with small dimensions that reduce the volume in which a cell may be located, they are especially preferred for use with acoustic ejection as locating a cell suitable for ejection is greatly simplified. For example, for a cell type or mixture of cell types having relatively uniform size, for example mean diameter of 10.0 μm, SD≈0.5 μm, the channel can be engineered to be about 12.0 μm wide, effecting a single file of cells uniformly a mean distance of about 1.0 μm from the fluid surface (ejection volume≈4/3πr³=0.52 pL), without for example providing a ramp (FIG. 5D) or otherwise promoting a short distance between surface and cell location as by the preceding methods that effect a net upwards force on the cells. The channel depth is as appropriate for desired fluid flow in the channel, but is preferably equipped with a means for directing cells to a position sufficiently close to the surface for ejection, which may comprise a channel depth no more than ten times cell diameter or dimension. Specifically employed are 40 μm deep channels with a ramp like structure directing the cells to the top with a ramp height of about 25 μm. The cells can be ejected from the channel at a certain limited distance range along the fluid flow axis, reducing the area of fluid surface scanned. For example a 50 μm aperture for ejecting cells can be provided in a closed capillary, or a limited distance along the flow axis of an open capillary may be used for ejection, a significant advantage being that the cells move past the ejector, reducing the area scanned for cells. Even when employing such methods to float cells in a macro-scale container such as a petri dish, significant amounts of time will be wasted scanning in the plane parallel to the fluid surface to locate a cell to eject. The advantages of employing microfluidic channels are only slightly diminished for a wider range of cell sizes for example, red blood cells (RBC, mean diameter of 7 μm, SD≈0.3 μm, biconcave disc, height≈3 μm) mixed with the preceding cell type (mean diameter of 10.0 μm, SD≈0.5 μm). Although the RBCs can be a significant depth from the surface relative to the fluid ejection volume and corresponding energy required to eject a RBC, this can be overcome by the described methods of forcing cells toward the fluid surface, and the advantage of limiting the lateral search to about 12 μm width as opposed to several cm wide petri dish is immediately apparent.

Once a cell sufficiently close to the surface is located and determined to meet any other criteria for ejection, the acoustic radiation generator is activated to produce an acoustic wave that is focused by the focusing means to direct the acoustic wave to a focal point 47 near fluid surface 17, with the amount of energy being sufficient to eject a volume of fluid substantially corresponding to the volume of the cell or cells to be ejected so that any ejected volume does not contain more than one cell. The precise amount of energy required to eject only the required volume and no more can be initially calibrated by slowly increasing the energy applied from an amount insufficient to eject a cell desired for ejection until there is just enough energy applied to eject the cell the desired distance to the targeted substrate locale. After this initial calibration approximately the same energy, with adjustment for any change in fluid level, may be applied to eject cells of substantially the same volume as the initial calibration cell. As a result, droplet 49, containing a single living cell, is ejected from fluid surface 17 into the first substrate well 55 of the substrate well plate 45. The cell containing droplet is retained on the substrate well plate by surface tension.

Then, as shown in FIG. 2C, the substrate well plate 45 is repositioned by a substrate positioning means 50 such that substrate well 57 is located directly over cell container or reservoir (well or channel) 15 in order to receive a cell containing droplet therefrom. FIG. 2C also shows that the ejector 33 has been repositioned by the ejector positioning means below cell container well 15 to acoustically couple the ejector and the container through acoustic coupling medium 41. Since the substrate well plate and the reservoir well plate or channels on a planar substrate are differently sized, there is only correspondence, not identity, between the movement of the ejector positioning means and the movement of the substrate well plate. Once properly aligned as shown in FIG. 2C, the acoustic radiation generator 35 of ejector 33 is activated to produce an acoustic wave that is then directed by focusing means 37 to a focal point near the fluid surface 19 for detection of the presence of a cell sufficiently close to the carrier fluid surface for ejection. After detection and measurement of any property forming a criterion for ejection, the acoustic radiation generator 35 of ejector 33 is activated to produce an acoustic wave that is then directed by focusing means 37 to a focal point near the fluid surface 19 from which cell containing droplet 53 is ejected onto the second well of the substrate well plate. It should be evident that such operation is illustrative of how the employed device may be used to transfer a plurality of single cells contained in appropriately sized droplets from one well plate to another of a different size. One of ordinary skill in the art will recognize that this type of transfer may be carried out even when the cells, the carrier fluid and both the ejector and substrate are in continuous motion. It should be further evident that a variety of combinations of reservoirs, well plates and/or substrates may be used in the employed device to transfer fluid droplets containing single cells. It should be still further evident that any reservoir may be filled with a fluid carrier or cells suspended in a fluid carrier through acoustic ejection of cell containing or cell free fluid droplets respectively prior to deploying the reservoir for further transfer of fluid droplets containing cells, e.g., for cell array deposition.

As discussed above, either individual, e.g., removable, reservoirs (well or channel) or plates (well or channel) may be used to contain cell suspensions in carrier fluids that are to be ejected, wherein the reservoirs or the wells of the well plate are preferably substantially acoustically indistinguishable from one another. Also, unless it is intended that the ejector is to be submerged in the fluid to be ejected, the reservoirs or well plates must have acoustic transmission properties sufficient to allow acoustic radiation from the ejector to be conveyed to the surfaces of the fluids to be ejected. Typically, this involves providing reservoirs or well bases that have appropriate acoustic impedance relative to the carrier fluid and are sufficiently thin relative to the acoustic attenuation of the material from which they are made, to allow acoustic radiation to travel therethrough without unacceptable dissipation or reflection. In addition, the material used in the construction of reservoirs must be compatible with the contained carrier fluids, and non-toxic to the suspended cells.

Thus, as it is intended that the reservoirs or wells contain live cells suspended in an aqueous carrier fluid, reservoirs made from materials that dissolve or swell in water or release compounds toxic to living cells into the aqueous carrier would be unsuitable for use in forming the reservoirs, substrates or well plates employed in the instant invention. For water-based fluids, a number of materials are suitable for the construction of reservoirs and include, but are not limited to, materials used in tissue or cell culture, biomaterials, mono or poly crystalline Si ceramics such as silicon oxide and aluminum oxide, metals such as stainless steel and platinum, and polymers such as polyester and polytetrafluoroethylene, and any preceding material functionalized on the surface. These materials may be prepared so that substances toxic to cells do not leach into the carrier fluid sufficient amounts to render the carrier fluid toxic to the cells, and so that their surface properties are appropriate for the intended use, for example containers or reservoirs from which cells are ejected may be surface functionalized to prevent cell adhesion to the solid wall or floor of the container material. Many well plates suitable for use with the employed device are commercially available and may contain, for example, 96, 384 or 1536 wells per well plate. Manufactures of suitable well plates for use in the employed device include Corning Inc. (Corning, N.Y.) and Greiner America, Inc. (Lake Mary, Fla.). However, the availability such commercially available well plates does not preclude manufacture and use of custom-made well plates containing at least about 10,000 wells, or as many as 100,000 wells or more. For array forming applications, it is expected that about 100,000 to about 4,000,000 reservoirs may be employed. In addition, to reduce the amount of movement needed to align the ejector with each reservoir or reservoir well, it is preferable that the center of each reservoir is located not more than about 1 centimeter, preferably not more than about 1 millimeter and optimally not more than about 0.5 millimeter from the center of the nearest neighbor reservoir.

Generally, the device may be adapted to eject fluids of virtually any type and amount desired. Ejected fluid may be aqueous and/or nonaqueous, but only aqueous fluids are compatible with transfer of living cells. Examples of aqueous fluids include water per se and water solvated ionic and non-ionic solutions and suspensions or slurries of solids, gels or discrete cells in aqueous liquids. Because of the precision that is possible using the inventive technology, the device may be used to eject droplets from a reservoir adapted to contain no more than about 100 nanoliters of fluid, preferably no more than 10 nanoliters of fluid. In certain cases, the ejector may be adapted to eject a droplet from a reservoir adapted to contain about 1 to about 100 nanoliters of fluid. This is particularly useful when the fluid to be ejected contains rare or expensive biomolecules or cells, wherein it may be desirable to eject droplets having a volume of about up to 1 picoliter.

From the above, it is evident that various components of the device may require individual control or synchronization to form an array of cells on a substrate. For example, the ejector positioning means may be adapted to eject droplets from each cell container or reservoir in a predetermined sequence associated with an array to be prepared on a substrate surface. Similarly, the substrate positioning means for positioning the substrate surface with respect to the ejector may be adapted to position the substrate surface to receive droplets in a pattern or array thereon. Either or both positioning means, i.e., the ejector positioning means and the substrate positioning means, may be constructed from, e.g., levers, pulleys, gears, linear motors a combination thereof, or other mechanical means known to one of ordinary skill in the art. It is preferable to ensure that there is a correspondence between the movement of the substrate, the movement of the ejector and the activation of the ejector to ensure proper pattern formation.

Moreover, the device may include other components that enhance performance. For example, as alluded to above, the device may further comprise cooling means for lowering the temperature of the substrate surface to ensure, for example, that the ejected droplets adhere to the substrate, and rapidly freeze the cells to maintain their viability. The cooling means may be adapted to maintain the substrate surface at a temperature that allows fluid to partially or preferably completely freeze shortly after the cell containing fluid droplet comes into contact therewith. In the case of aqueous fluid droplets containing cells, the cooling means should have the capacity to maintain the substrate surface at no more than about 0° C., preferably much colder. In addition, repeated application of acoustic energy to a reservoir of fluid may result in heating of the fluid. Heating can of course result in unwanted effects on living cells. Thus, the device may further comprise means for maintaining fluid in the cell containers or reservoirs at a constant temperature. Design and construction of such temperature maintaining means are known to one of ordinary skill in the art and may comprise, e.g., components such a heating element, a cooling element, or a combination thereof. For biomolecular and live cell deposition applications, it is generally desired that the fluid containing the biomolecule or cells is kept at a constant temperature without deviating more than about 1° C. or 2° C. therefrom. In addition, for live cells, it is preferred that the fluid be kept at a temperature that does not exceed about 1° C. above the normal temperature from which the cell is derived in the case of warm blooded organisms, and at about 16° C.± about 1° C. for all other organisms whether prokaryotic or eukaryotic, except, for all organisms, in the case that the specific cell type is known to have poor viability unless chilled. Cells that require chilling for viability will be appreciated by those of ordinary skill in the art of culturing and maintaining cells to require a saline carrier fluid of appropriate osmolality (slightly hyperosmotic) at about −1° C.± about 1° C. Thus, for example, when the biomolecule-containing fluid is aqueous, it may be optimal to keep the fluid at about 1° C.± about 0.5° C. during ejection.

The invention may involve modification of a substrate surface prior to acoustic ejection of cell containing fluid droplets thereon. Surface modification may involve functionalization or defunctionalization, smoothing or roughening, coating, degradation, passivation or otherwise altering the surface's chemical composition or physical properties. In one embodiment the invention requires functionalization with a cognate moiety to an externally displayed marker moiety, but other surface modifications described may affect the success of the inventive method in a specific context

One such surface modification method involves altering the wetting properties of the surface, for example to facilitate confinement of a cell contained in a droplet ejected onto the surface within a designated area or enhancement of the kinetics for the surface attachment of molecular moieties for functionalizing the substrate or a specific substrate locale, as by patterning biotinylation by acoustic ejection of a biotinylating solution. A preferred method for altering the wetting properties of the substrate surface involves deposition of droplets of a suitable surface modification fluid at each designated site of the substrate surface prior to acoustic ejection of fluids to form an array thereon. In this way, the “spread” of the acoustically ejected droplets and contained cells may be optimized and consistency in spot size (i.e., diameter, height and overall shape) ensured. One way to implement the method involves acoustically coupling the ejector to a modifier reservoir containing a surface modification fluid and then activating the ejector, as described in detail above, to produce and eject a droplet of surface modification fluid toward a designated site on the substrate surface. The method is repeated as desired to deposit surface modification fluid at additional designated sites. Similarly by the methods of copending applications (“Focused Acoustic Energy in the Preparation of Combinatorial Composition of Matter Libraries” U.S. Ser. No.______, inventors Mutz and Ellson, filed on even date herewith, and “Focused Acoustic Energy in the Preparation of Peptidic Arrays,” U.S. Ser. No. 09/669,997, inventors Mutz and Ellson, filed on Sep. 25, 2000, both of which are assigned to Picoliter, Inc. (Cupertino, Calif.)) or by other methods of generating arrays of biomolecules attached or linked to a substrate surface, cognate moieties that specifically bind to marker moieties displayed on the surface of transformed or untransformed cells may be patterned on the substrate surface. Alternatively a single cognate moiety such as biotin can be linked to the substrate surface either uniformly, or in a pattern, such as biotinylated areas surrounded by non-biotinylated areas, and the cells to be patterned can be transformed to display streptavidin on their surface.

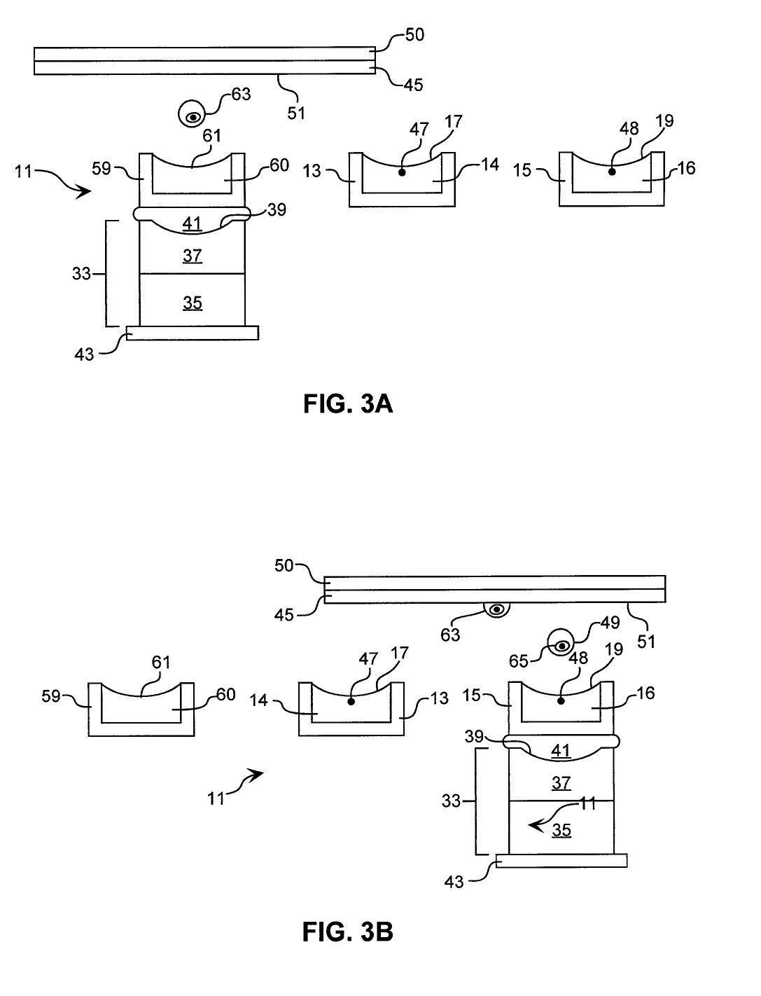

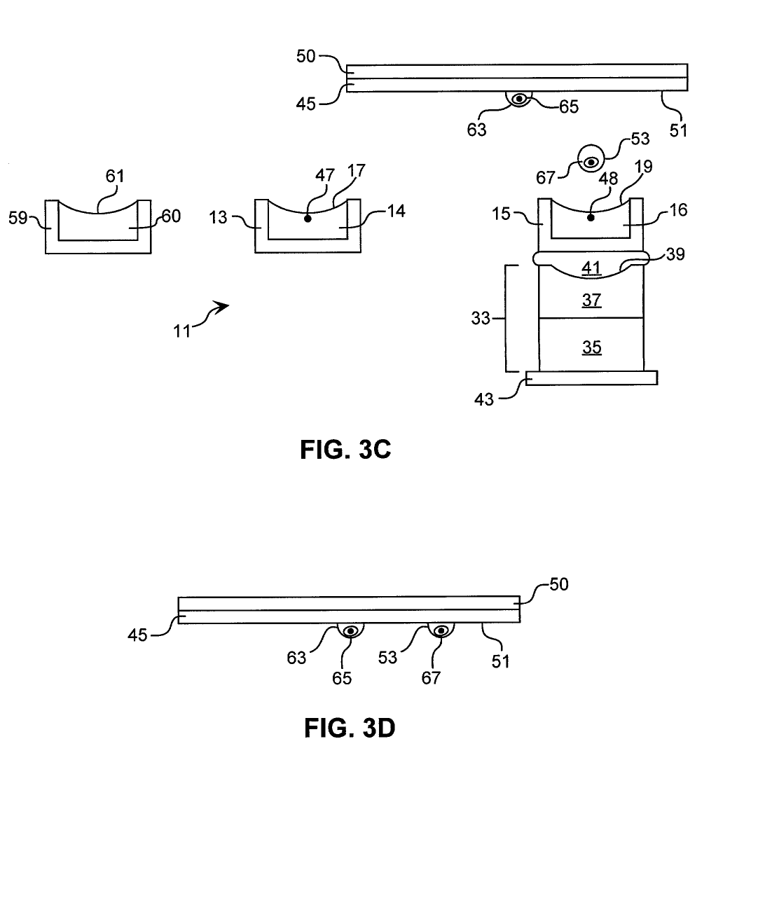

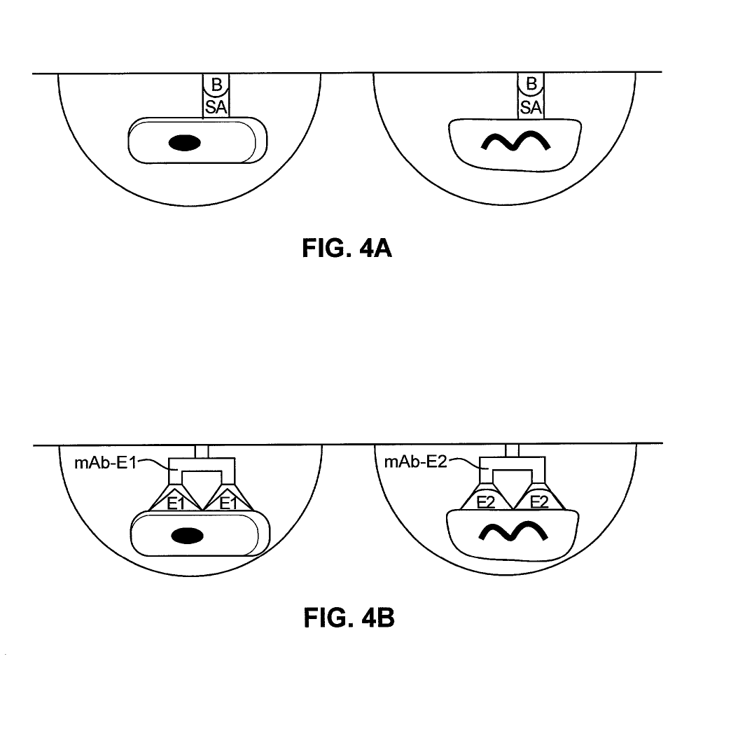

FIG. 3 schematically illustrates in simplified cross-sectional view a specific embodiment of the aforementioned method in which a two cells are deposited at different siteson a substrate using a device similar to that illustrated in FIG. 1, but including an additional reservoir 59, which may contain a different type of cell, or may contain a surface modification fluid, the fluid 60 having a fluid surface 61. FIG. 3A illustrates the ejection of a droplet 63 (here depicted containing a cell rather than a surface modification fluid) of surface modification fluid or carrier fluid containing cells, 60. When desired, a surface modifier may be employed for various purposes, for example a surface modifier may be selected to alter the wetting properties of designated sites on surface 51 of the substrate 45 where the cells are to be deposited. The ejector 33 is positioned by the ejector positioning means 43 below modifier reservoir 59 in order to achieve acoustic coupling therewith through acoustic coupling medium 41. Substrate 45 is positioned above the modifier reservoir 19 at a location that enables acoustic deposition of a droplet of surface modification fluid 60 at a designated site. Once the ejector 33, the modifier reservoir 59 and the substrate 45 are in proper alignment, the acoustic radiation generator 35 is activated to produce acoustic radiation that is directed by the focusing means 37 in a manner that enables ejection of droplet 63 of the surface modification fluid 60 from the fluid surface 61 onto a designated site on the underside surface 51 of the substrate. Once the droplet 63 contacts the substrate surface 51, the droplet modifies an area of the substrate surface to result in an increase or decrease in the surface energy of the area with respect to deposited fluids.

Then, as shown in FIG. 3B, the substrate 45 is repositioned by the substrate positioning means 50 such that the region of the substrate surface modified by droplet 63 is located directly over reservoir 13. FIG. 3B also shows that the ejector 33 is positioned by the ejector positioning means below reservoir 13 to acoustically couple the ejector and the reservoir through acoustic coupling medium 41. Once properly aligned, the ejector 33 is again activated so as to eject droplet 49 onto substrate. Droplet 49 contains a single cell 65, preferably displaying a marker moiety on its external cell membrane that is specifically bound by a cognate moiety linked to the surface to effect specific attachment to the surface. The marker moiety may occur in an untransformed cell or may be the result of transformation or genetic manipulation, and may optionally signify transformation to express a gene other than the marker, e.g. as a reporter of transformation with another gene.

Then, as shown in FIG. 3C, the substrate 45 is again repositioned by the substrate positioning means 50 such that a different site than the site having the first single cell 65 attached thereto is located directly over reservoir 15 in order to receive a cell contained in a droplet therefrom. FIG. 3B also shows that the ejector 33 is positioned by the ejector positioning means below reservoir 15 to acoustically couple the ejector and the reservoir through acoustic coupling medium 41. Once properly aligned, the ejector 33 is again activated so that droplet 53 is ejected onto substrate. Droplet 53 contains a second single cell.

Often cognate moieties are ligands including oligonucleotides and peptides. Marker moieties are likely to be peptides or peptidoglycans. The chemistry employed in synthesizing substrate-bound oligonucleotides can be adapted to acoustic fluid droplet ejection (see co-pending patent application U.S. Ser. No. 09/669,996, entitled “Acoustic Ejection of Fluids from a Plurality of Reservoirs,” inventors Ellson, Foote and Mutz, filed on Sep. 25, 2000 and assigned to Picoliter, Inc. (Cupertino, Calif.)). These methods may be used to create arrays of oligonucleotides on a substrate surface for use with the instant invention. Such adaptation will generally involve now-conventional techniques known to those skilled in the art of nucleic acid chemistry and/or described in the pertinent literature and texts. See, for example, DNA Microarrays: A Practical Approach, M. Schena, Ed. (Oxford University Press, 1999). That is, the individual coupling reactions are conducted under standard conditions used for the synthesis of oligonucleotides and conventionally employed with automated oligonucleotide synthesizers. Such methodology is described, for example, in D. M. Matteuci et al. (1980) Tet. Lett. 521:719, U.S. Pat. No. 4,500,707 to Caruthers et al., and U.S. Pat. Nos. 5,436,327 and 5,700,637 to Southern et al. Focused acoustic energy may also be adapted to in situ combinatorial oligonucleotide, oligopeptide and oligosaccharide syntheses for forming combinatorial arrays for use with the instant invention (see co-pending patent application U.S. Ser. No.______, entitled “Focused Acoustic Energy in the Preparation and Screening of Combinatorial Composition of Matter Libraries,” inventors Mutz and Ellson, referenced supra).

Alternatively, an oligomer may be synthesized prior to attachment to the substrate surface and then “spotted” onto a particular locus on the surface using the methodology of the invention. Again, the oligomer may be an oligonucleotide, an oligopeptide, oligosaccharide or any other biomolecular (or nonbiomolecular) oligomer moiety. Preparation of substrate-bound peptidic molecules, e.g., in the formation of peptide arrays and protein arrays, is described in co-pending patent application U.S. Ser. No. 09/669,997 (“Focused Acoustic Energy in the Preparation of Peptidic Arrays”), inventors Mutz and Ellson, filed on Sep. 25, 2000 and assigned to Picoliter, Inc. (Cupertino, Calif.). Preparation of substrate-bound oligonucleotides, particularly arrays of oligonucleotides wherein at least one of the oligonucleotides contains partially nonhybridizing segments, is described in co-pending patent application U.S. Ser. No. 09/669,267 (“Arrays of Oligonucleotides Containing Nonhybridizing Segments”), inventor Ellson, also filed on Sep. 25, 2000 and assigned to Picoliter, Inc.

These acoustic ejection methods enable preparation of molecular arrays, particularly biomolecular arrays, having densities substantially higher than possible using current array preparation techniques such as photolithographic processes, piezoelectric techniques (e.g., using inkjet printing technology), and microspotting, for use with the instant invention. The array densities that may be achieved using the devices and methods of the invention are at least about 1,000,000 biomolecules per square centimeter of substrate surface, preferably at least about 1,500,000 per square centimeter of substrate surface. The biomolecular moieties may be, e.g., peptidic molecules and/or oligonucleotides. Often such densities are not necessary for creating sites containing individual cells, which are separated by a distance from other cells. But adaptation of such methods, for example, to functionalize a discrete portion of a site surface with cognate moieties which specifically bind a marker moiety, may be useful in localizing the cells within the site, or for situations where the cells are deliberately arrayed in close proximity. For example, for a lymphocyte array (D: small˜8 μm, medium˜12 μm, large˜14 μm), when the sites are 100 μm×100 μm squares, functionalizing a 10 μm diameter spot in the center of each site with the appropriate cognate moiety to specifically bind the spotted cell will ensure sufficient cell separation to allow, for example, testing or screening of individual cells. The testing may be performed, for example, by acoustic deposition of reagent containing fluid droplets of sufficient volume to expose or treat the cell without necessarily exposing cells at adjacent sites to the same condition, permitting, for example, combinatorial screening of cells.

It should be evident, then, that many variations of the invention are possible. For example, each of the ejected cell containing droplets may be deposited as an isolated and “final” feature. Alternatively, or in addition, a plurality of ejected droplets, each containing one or a plurality of cells may be deposited on the same location of a substrate surface in order to synthesize a cell array where each site contains multiple cells of either known or unknown but ascertainable number, or to pattern cells for other purposes such as tissue engineering on a pattern replicating a specific histologic architecture. For cell array and patterning fabrication employing attachment, it is expected that washing steps may be used between droplet ejection steps. Such wash steps may involve, e.g., submerging the entire substrate surface on which cells have been deposited in a washing fluid.

The invention enables ejection of droplets at a rate of at least about 1,000,000 droplets per minute from the same reservoir, and at a rate of at least about 100,000 drops per minute from different reservoirs. In addition, current positioning technology allows for the ejector positioning means to move from one cell container or reservoir to another quickly and in a controlled manner, thereby allowing fast and controlled ejection of different fluids. That is, current commercially available technology allows the ejector to be moved from one reservoir to another, with repeatable and controlled acoustic coupling at each reservoir, in less than about 0.1 second for high performance positioning means and in less than about 1 second for ordinary positioning means. A custom designed system will allow the ejector to be moved from one reservoir to another with repeatable and controlled acoustic coupling in less than about 0.001 second. In order to provide a custom designed system, it is important to keep in mind that there are two basic kinds of motion: pulse and continuous. Pulse motion involves the discrete steps of moving an ejector into position, emitting acoustic energy, and moving the ejector to the next position; again, using a high performance positioning means with such a method allows repeatable and controlled acoustic coupling at each reservoir in less than 0.1 second. A continuous motion design, on the other hand, moves the ejector and the reservoirs continuously, although not at the same speed, and provides for ejection during movement. Since the pulse width is very short, this type of process enables over 10 Hz reservoir transitions, and even over 1000 Hz reservoir transitions.

In order to ensure the accuracy of fluid ejection, it is important to determine the location and the orientation of the fluid surface from which a droplet is to be ejected with respect to the ejector. Otherwise, ejected droplets may be improperly sized or travel in an improper trajectory. Thus, another embodiment of the invention relates to a method for determining the height of a fluid surface and the proximity of a cell in a reservoir between ejection events. The method involves acoustically coupling a fluid-containing reservoir to an acoustic radiation generator and activating the generator to produce a detection acoustic wave that travels to the fluid surface and is reflected thereby as a reflected acoustic wave. Parameters of the reflected acoustic radiation are then analyzed in order to assess the spatial relationship between the acoustic radiation generator and the fluid surface. Such an analysis will involve the determination of the distance between the acoustic radiation generator and the fluid surface and/or the orientation of the fluid surface in relationship to the acoustic radiation generator.

More particularly, the acoustic radiation generator may activated so as to generate low energy acoustic radiation that is insufficiently energetic to eject a droplet from the fluid surface. This is typically done by using an extremely short pulse (on the order of tens of nanoseconds) relative to that normally required for droplet ejection (on the order of microseconds). By determining the time it takes for the acoustic radiation to be reflected by the fluid surface back to the acoustic radiation generator and then correlating that time with the speed of sound in the fluid, the distance—and thus the fluid height—may be calculated. The presence and depth of a cell beneath the surface can be determined likewise. Of course, care must be taken in order to ensure that acoustic radiation reflected by the interface between the reservoir base and the fluid is discounted. It will be appreciated by those of ordinary skill in the art that such a method employs conventional or modified sonar techniques.

Once the analysis has been performed, an ejection acoustic wave having a focal point substantially the center of a cell near the fluid surface is generated in order to eject at least one droplet of the fluid, wherein the optimum intensity and directionality of the ejection acoustic wave is determined using the aforementioned analysis optionally in combination with additional data. The “optimum” intensity and directionality are generally selected to produce droplets of consistent size and velocity. For example, the desired intensity and directionality of the ejection acoustic wave may be determined by using not only the spatial relationship assessed as above, but also geometric data associated with the reservoir, fluid property data associated with the fluid to be ejected, cell dimensions and consequent cell volume, and/or by using historical cell containing droplet ejection data associated with the ejection sequence. In addition, the data may show the need to reposition the ejector so as to reposition the acoustic radiation generator with respect to the fluid surface, in order to ensure that the focal point of the ejection acoustic wave is near the fluid surface, where desired. For example, if analysis reveals that the acoustic radiation generator is positioned such that the ejection acoustic wave cannot be focused near the fluid surface, the acoustic radiation generator is repositioned using vertical, horizontal and/or rotational movement to allow appropriate focusing of the ejection acoustic wave.

Because one aspect of the invention is ejection of a single cell, the selective nature of the invention will be immediately appreciated. Using simple ejection, cells of sufficiently different size can be separated, starting with ejection of the smallest cells and this can be employed as a type of cell sorter in addition to a method for making arrays. For example because monocytes (D≈20 μm) are much larger than both small (D≈8 μm) and medium and large lymphocytes (D≈12-14 μm), corresponding to a cellular volume for monocytes of about 3 times (large lymphocytes) to about 16 times (small lymphocytes) greater in size and volume. A mixture of these cells may be selectively ejected for arraying or sorting. The minimum acoustic energy level adequate to eject small lymphocytes will be insufficient to eject the large lymphocytes that are approximately 5 times as voluminous and massive and monocytes which are approximately 16 times as voluminous and massive than the small lymphocytes respectively.

Once all the small lymphocytes have been ejected the large lymphocytes may be ejected using minimum acoustic energy level adequate to eject large lymphocytes (which will be adequate for ejecting medium lymphocytes) with little danger of ejecting monocytes, which are approximately 3 times as voluminous and massive. Surface functionalization with cognate moieties to marker moieties inherently or by transformation displayed externally on a cell exterior offers another level of selectivity, albeit requiring ejection onto a surface. Finally, as the invention provides for acoustic location of a cell to determine whether it is close enough to the surface to be ejected, various properties may be measured and used as additional criteria for ejection. One of skill in the art of cell sorting will appreciate that such ejection with additional criteria can be adapted to traditional cell sorting applications by ejection in a trajectory appropriate to transfer the ejected cell to another fluidic container, or by spotting onto a substrate and subsequently washing the desired cells into a container as desired. Likewise, the invention adapted to sorting circumscribed volumes such as cells having different acoustic impedance than the carrier fluid in which immediately will be appreciated to be adaptable to sorting particles (such as glass or polymer beads), including particles tagged with a specific moiety or particles which may be intrinsically evaluated by measurement of some property. Properties useful in sorting both cells and other circumscribed volumes differing in acoustic impedance, such as solid or gel particles, from the carrier fluid including acoustic density and/or size both of which can be measured by known acoustic means detecting acoustic waves reflected by the interface between the circumscribed volume and carrier fluid to permit acoustic density calculation from the reflection coefficient, and sonar imaging methods to determine size and shape.

The instant invention embodied as a cell sorter is preferably employed with at least one channel, preferably more than one channel, from which cells are ejected. An ejection channel preferably allows cells that are to be sorted to pass in single file. Cells may be ejected to other types of containers, including a fluidic channel, or onto a substrate not having physical separations such as a planar array where the cells are localized by attachment at sufficient distances from one another to form a virtual separate container for each cell, or the substrate may have some cells arrayed close enough to permit interactions between some of the cells in the virtual containers. Conventional containers such as an array of wells on a commercial well plate may serve as physical containers for an array of virtual containers of one or more cells, or may serve as receptacles for individual or multiple cells of one or more cell population. Mixtures of cells such as monocytes, B lymphocytes and T lymphocytes may be desired for experiment.

Multiple ejectors for each ejection channel can increase throughput, especially with multiple channels, and this is preferred. Each ejector may be coordinated with one or more sensing or detecting means. Such coordination may be effected manually as by an individual operating the one or plurality of ejectors associated with a given ejection channel or preferably as part of an integrated system employing a processor to integrate the detection and ejection to make the selection based upon inputted parameters for the detected property. Different ejection channels may be designed for different sized cell populations, which can be separated by conventional means according to size, including enrichment techniques and absolute filtration. The preferable multiple ejectors for each ejection channel may eject cells to multiple targets such as multiple well plates or different wells in the same plate, or different containers including fluid channels.

Target channels for carrying selectively ejected cells to a destination, may be provided. For convenient ejection of cells from an ejection channel open on top in at least one region into efferent or target channels or containers open on top and located nearby, the acoustic ejection means or source of focused acoustic energy should be capable of imparting a non-vertical trajectory (velocity component parallel to the fluid surface) to the ejected droplet, e.g., by rendering the ejected droplet with a non-vertical velocity component in the ejection. It will readily be appreciated that such a non-vertical ejection velocity, if not parallel to the flow in the ejection channel can eject a droplet into a container such as a target channel that is horizontally spaced from the ejection channel. Such a target channel for receipt of cells from an ejection channel may or may not be in fluidic contact with the ejection channel; further the target channel for receiving an acoustically ejected cell or cells may also serve as an ejection channel for ejection of the cell to another target channel or container, including the channel from which the cell was originally ejected.

For maximum separation efficiency and flexibility, the horizontal or surface parallel component of the ejection velocity may be varied to permit ejection of a cell vertically from the fluid or with a sole non-vertical component of velocity imparted by the flow in the ejection channel and parallel to the flow therein, or with various non-vertical velocity components which are not parallel to the direction of fluid flow in the channel. Such directionality of ejection that is controllable by the focusing of the acoustic ejector itself permits, for example, ejection by one ejector from a central ejection channel to either of two target channels flowing on either side of the ejection channel, each target channel flowing in substantially the parallel or anti-parallel direction to the flow of the ejection channel in the region of such a single directable acoustic ejector. Most preferably such an adjustable ejection trajectory acoustic ejector may be moved to eject cells from one channel to another, for example in the three channel arrangement where cells are ejected to one or the other laterally spaced channels from a central channel, the ejector may be preferably moved to either lateral channel to eject cells either back into the central channel, to the opposite laterally spaced channel from the central channel, or to other channels than the three described above that are sufficiently near the lateral channels.

Additionally it will be readily appreciated that the ejection or target container or channel need not flow in any specific direction, or at all, either absolutely or relative to the other container or channel during ejection. If flowing the target channels may loop towards the ejection channel or the ejection channel may loop towards a target channel; alternatively target or ejection channels may originate (with an appropriate source for cells suspended in carrier fluid originating from, for example, above or below the channel floor or top) or the target or ejection channels may cross over or under each other as may be conveniently fabricated by routine microfabrication methods (see, for example, U.S. Pat. No. 6,044,981 to Chu et al. teaching nanometer scale buried channel filter constructed using sacrificial oxide and standard photolithographic techniques by layering of a Si material).

For clinical cell sorting applications where speed, high throughput are desired for therapeutic purposes limited sorting is often possible. For example, only certain specific cell need be separated for infusion in the case of heterologous allografts (or possibly xenografts) of cells such as immature stem cells which are less likely to mount a graft versus host response when used to replace cells after irradiation or chemotherapy to ablate cells in a patient as in cancer treatment. Or for autologous reinfusion (reinfused cells necessarily allografts) only diseased cells need be removed before for reinfusing. In such cases the multiple acoustic cell ejection means pre channel may be used to eject the undesired cells from a given channel sequentially or in series as coordinated with the single or plurality of detecting means to increase the cell throughput per channel. This increased throughput per channel is in addition to combination of multiple channels for ejection in parallel.

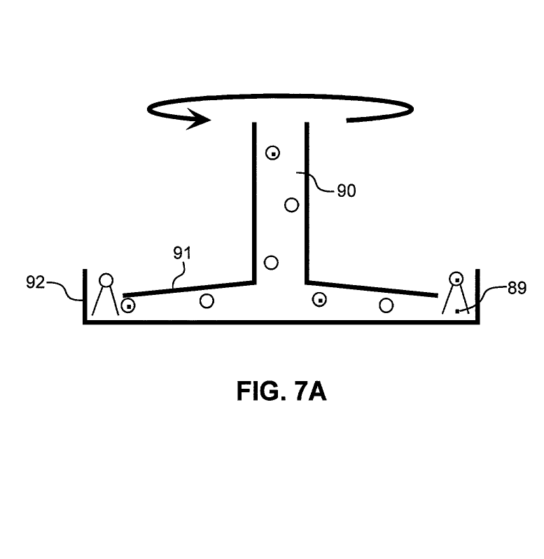

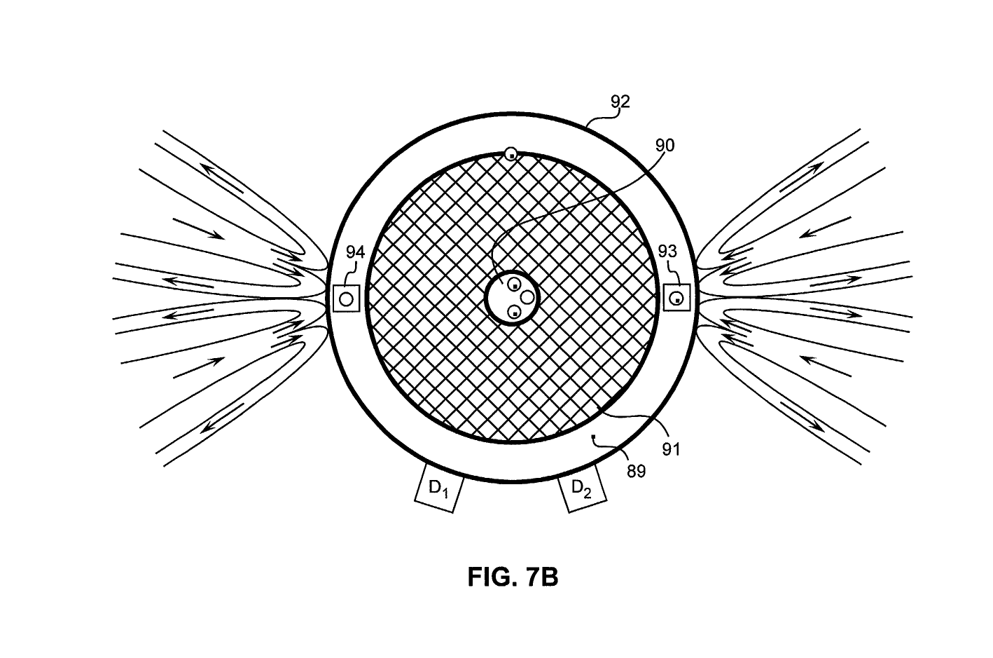

FIG. 7 depicts a device having a central fluidic channel that feeds cells with high throughput laterally to a peripheral channel from which the cells are ejected onto the substrate, preferably by use of multiple ejectors. FIG. 7A illustrates a side view of a vertical channel containing cells within a larger vessel. The periphery of the larger vessel is fluidically accessible from the vertical channel only by passing under an angled lip projecting laterally from the vertical channel with the distance between the lip and the floor of the larger vessel decreasing radially outward so that cells can pass radially outwards from the central channel, to the periphery.

At the periphery a channel is formed where cells are spaced further apart and move in the horizontal plane, relative to spacing in the vertical channel. FIGS. 7A and 7B depict two focused acoustic elements at two ejection sites, 93 and 94, located at the outer circumference of the rotating fluid chamber for ejecting cells that reach a peripheral channel, 89, located just inside the container wall, 92. A collecting device or substrate is not shown. Multiple focused acoustic elements are preferably placed on the circumference, with each preferably preceded by at least one cell property detector, here D₁ and D₂. Liquid can also be drawn from above the angled lip 91 to further induce particle flow to the ejection zone at the focal spot of the acoustic element, and decrease the horizontal area in which a cell may be present. This configuration has the advantage of sweeping a large volume of fluid into the ejection zone and increasing both throughput and overall efficiency of cell sorting. The entire volume of fluid and all cells contained pass through a common central channel, 90, prior to passing under angled lip 90 en route to the peripheral channel 89. Excess fluid entering channel 89 may be removed by acoustic ejection (not shown), or by conventional microfluidic channels having a dimensions too small for the cells that are separated to pass through, such channels will be appreciated as readily made by routine microfabrication techniques.

In addition to multiplexing detectors and ejectors in one such separation unit, multiple such units may be simultaneously employed in parallel to greatly enhance throughput and efficiency. Furthermore, non-binary ejection decisions may be made at each ejector in the unit, and further flexibility may be obtained by employing units in series for complex separations. Units employed in series, may be optimized for successively different mean cell size or other cell parameters for complex sorting procedures.

The preferred directable acoustic ejection means can be adjusted to render the ejected droplet with a vertical ejection despite channel motion by ejecting the droplet with a horizontal velocity relative to the flow of fluid in the channel of exactly equal and in an opposite direction than the fluid flow, or can as easily eject the droplet with a net horizontal velocity in a direction perpendicular to the fluid flow in the channel, whether or not there is any horizontal velocity relative to a stationary frame of reference in the axis parallel to the fluid flow of the channel. Thus, for example, a droplet containing a cell may be ejected from one directable ejector to one of two channels near the ejection channel, or onto a substrate surface disposed above the ejection channel. The ejection is non-binary because rather than ejecting or not ejecting, four choices exist: not ejecting, ejecting to two possible channels and ejecting to the substrate surface. Similarly even without the solid substrate as a possible target for ejection, the choices of not ejecting or ejecting to either of the two target channels provide a ternary rather than binary selection scheme at a single ejector.

Various detection means are routinely employed, often using tags such as specific Abs which are imparted with some property such as ferromagnetic or fluorescent properties and the like. Such tagged and intrinsic properties, such as intrinsic fluorescent properties can yield various properties such as diameter, volume ratio of nuclear volume to cytoplasmic volume and, in some cases intracytoplasmic and intranuclear conditions. For example measuring intrinsic fluorescence of the amino acid tryptophan (Trp) can yield valuable information as to e cells identity by detecting contributions to the net spectrum from specific proteins or of the same proteins under different conditions. Each tryptophan will have absorption and emission spectra that are affected or shifted by the local environment in the protein. Thus one protease will have different spectra than another protease, and the same protease will experience a shift in its spectra if the pH of the fluid surrounding it is changed. Thus among granulocytes, for example, the neutrophils or polymorphonuclear cells (PMNs), with their plethora of neutrophilic membrane surrounded granules, will exhibit a different net intrinsic Trp fluorescence that eosinophils and basophil, with their characteristically different granules, by virtue of different shifts in intrinsic Trp fluorescence of the same granule membrane proteins caused by differences in the granules and presence of different proteins in the granules themselves. Similarly nuclei, with high levels of densely packed histones in the chromatin will be discernable from cytoplasm, by intrinsic Trp fluorescence. Fluorescent tagging of the cells external surface permits sizing the cell by measuring fluorescence emission of cells flowing past a detector, with the duration of emission of any components of the emission spectrum through the detection window proportional to dimension in the cross section parallel to flow, and orthogonal to a line from detector to cell center, giving a signal proportional to cell diameter if measured at a level permitting detection of the longest possible signal, e.g. across the cell center or along the longest transecting distance of the cross section. If the detection is also of emission from all points in the cross sectional dimension orthogonal to both the flow direction and the axis from detector to cell center, integrating intensity of fluorescence over time will yield an integrated signal proportional to the presented cross sectional area. Differentiation of the intensity measured as a function of time with respect to time (which in turn corresponds to distance for constant velocity flow) yields some information on geometry with spherical cells expected to exhibit a less spiked signal than say cuboidal cells. If intrinsic fluorescence of cell contents is measured the duration of emission is proportional to the cell diameter, while the emission intensity integrated over time is proportional to total volume passing across the detection window and intensity differentiated with respect to time yields information on geometry.

It will be readily appreciated that any one of a number of different properties or parameters will be detected. Often the detected property will require a probing or excitation signal. For example most spectroscopic measurements including fluorescence, will measure an electromagnetic emission or absorption as a result of an excitation by electromagnetic waves. Acoustic or sonar type detection will require a probing signal of focused acoustic energy, and measure reflected acoustic energy that is reflected as a result of differences in acoustic impedance at an interface such as the cell surface carrier fluid interface or the interface between nucleus and cytoplasm. It will be appreciated, for example, that the differences in acoustic impedance between densely packed and tightly held nuclear material and looser, less dense cytoplasm will permit acoustic detection of nucleated cells and the ratio of nuclear to cytoplasmic volumes in a manner analogous to employing intrinsic fluorescence for example. A good example of now routine methods employing a laser beam having a diameter larger than the largest of the cells in a mixture of cells and measuring both light scatter and fluorescence for sizing surface fluorescent tagged cells is described in U.S. Pat. No. 4,765,737 to Harris et. al. That equivalent information may be derived from acoustic detection or sonar will be readily appreciated. But the adaptability of intrinsic fluorescence detection to measure volume of nucleus and cytoplasm will be appreciated to offer, when appropriately calibrated, more reliable estimate of total volume, cytoplasmic volume and nuclear volume than volumes extrapolated by acoustic measurement of dimensions of a given cross section. Further intrinsic fluorescence can, for example, distinguish between morphologically similar granulocytes which differ primarily in the types of membrane bounded granules, effecting a differently shifted net intrinsic Trp fluorescence signal for basophils, PMNs and eosinophils, and between different agranulocytes such as monocytes and lymphocytes.

More generally, the detected differences in physical characteristics may be differences in visual characteristics, detectable by the naked eye or under magnification, or to a video camera integrated with suitably programmed image processing equipment. Differences in optical characteristics such as transmissivity, reflectivity, color, polarization or the like may be measured. The differences detected may be of other physical characteristics such as electrical conductivity, capacitance, inductance, permeability to microwaves, magnetic properties ultrasound or acoustic energy, or spectroscopic techniques based upon other types of electromagnetic radiation or the like, as long as the measurement does not substantially affect cell viability.

Once separated according to a specific property, the cells can be separated again. For example a mixture of cells having a wide size distribution may be separated into three size bins: large, medium and small. These sized populations, for example the medium sized population, may each be separated again by size into three more bins. Often successive detection of smaller differences in the same property may be effected by changing detection conditions. For example, sub-groups of cells separated by gross differences in size in a rapidly moving channel by sonar or acoustic imaging may be separated further by size in more slowly moving channels. Blood cells are one example of cells which may be thus sorted. A mixture of monocytes (spheres˜11-20 μm diameter), lymphocytes (spheres˜8 [small], 12 [medium], 14 [large] μm diameter) and erythrocytes (doughnut like discs ˜7 μm diameter by ˜3 μm high) may be separated into monocytes, lymphocytes and erythrocytes by use of an ejection channel about 22 μm wide employing means for reliably floating all cells, such as an appropriate density carrier fluid that does not affect cell viability or a physical ramp-like structure in the ejection channel just upstream from the ejection site. Lymphocytes and monocytes are ejected into appropriately sized channels with the target channel for lymphocytes having a width of about 15 μm and the target channel for the monocytes having a width of up to about 22 μm. Some erythrocytes may be ejected with lymphocytes and are less likely to be ejected with monocytes, because of their ability to be positioned alongside the ejected cell during ejection in the ejection channel. The lymphocytes once flowing in the 15 μm wide channel may be sized acoustically again, using a slower rate of flow which is adequately rapid for maintaining overall throughput because the subpopulation of lymphocytes is merely a fraction of the total number of cells.

Alternatively different cells in a mixture can be separated by intrinsic fluorescence using excitation at a given frequency and measured emission per measured volume; with the initially separated groups being separated again by intrinsic fluorescence using excitation at a slightly shifted frequency and/or shifted frequency for measuring emission per measured volume, to distinguish shifted intrinsic fluorescence intracellular conditions, e.g., shifting of some or all of the intrinsic tryptophanyl fluorescence of specific cell populations, subpopulations or sub-subpopulations because of differences, such as the ratio of nucleus volume to cytoplasm volume, which differs, for example, between small medium and large lymphocytes, causing small lymphocyte intrinsic fluorescence to arise primarily from basic nuclear proteins with consequently shifted excitation (absorption) and emission frequencies.

The preceding is a type of serial multiplexing, and resembles serial multiplexing wherein different properties are measured for all or some of initially separated sub-populations to further separate them. This is also effectively closer to analog separation than binary to the extent that the same property is used, as more different, for example, size groups are generated along the continuum of sizes. Also readily appreciable is that any serial process may be carried out in parallel to increase throughput, and so offers another level of multiplexing.

For complex mixtures of cells both serial and parallel multiplexing is preferably combined with multiple detectors and different types of detectors. For example acoustic detection combined with intrinsic fluorescence at multiple detection sites. Many parameters may be determined from measuring both acoustic reflection and fluorescence over time and integration and differentiation thereof. For example cell and nuclear diameter, presence of nucleus, and nuclear/cytoplasmic/total cell volumes, dimension ratios, volume ratios, may be determined by integrating acoustic and intrinsic fluorescence data. For the purposes described herein the cytoplasmic volume is taken to include the volume of included organelles such as mitochondria in macrophages and the granules of granulocytes although these volumes are technically not cytoplasmic, and a more precise term would be extranuclear volume, being total cell volume minus nuclear volume. Measuring intrinsic fluorescence emissions at various frequencies, such as mean over cell types or weighted mean by representation of cell types in blood of Trp emission frequency intensity maximum, analogous mean for nuclei of nucleated blood cells, shift corresponding to PMN Trp emission frequency intensity maximum, shift corresponding to eosinophil Trp emission frequency intensity maximum, shift corresponding to basophil Trp emission frequency intensity maximum will allow distinguishing cells such as granulocytes that can not be distinguished by geometric parameters such as nuclear to cytoplasmic volume ratio. Although avoiding introduced tags will usually be desirable, any selection methods involving deliberately tagged cells can also be employed with the instant invention.

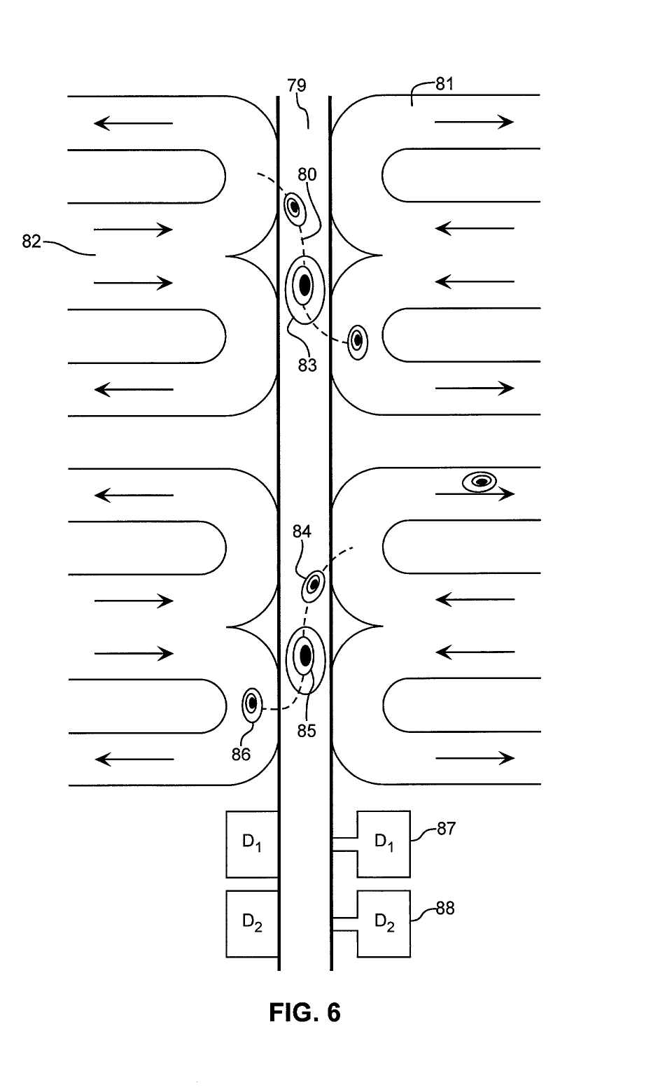

FIG. 6 depicts a top view of a central channel, an ejection channel, with two detecting devices D₁ and D₂ past which cells flow and two ejection sites, represented by large ellipses, each containing a depiction of a cell, from which cells may be ejected perpendicular to the surface onto a substrate (not shown), or into adjacent target channels. Cells flow past the detectors prior to reaching the ejection sites. Cells may be ejected from the ejection sites with the only velocity component being perpendicular to the plane substantially parallel to the fluid surface (here a horizontal plane, with the perpendicular thereto being vertical). When the perpendicular ejection velocity component is the only non-zero component of velocity, the ejection trajectory is perpendicular to the fluid surface (here vertical), permitting ejection onto a substrate surface (not shown here) for array formation as depicted in the preceding figures. Cell containing droplets may also be ejected with a non-perpendicular velocity component, permitting trajectories such as those depicted by dashed lines.

Channels depicted near the central or ejection channel are target channels for receiving ejected cells. At each side of the ejection site in the ejection channel, a common fluidic channel is divided into two channels just prior to reaching the ejection channel and the two channels loop towards the ejection channel, flowing parallel and antiparallel to the fluid flow in the ejection channel for a short distance, and sufficiently close to permit a cell to be ejected from the ejection channel to any selected target channel abutting the ejection channel near the ejection site. As configured in this depiction, a cell at one of the ejection sites may be selected not to be ejected, selected to be vertically ejected to a substrate surface, as to an array site on the substrate surface, or may be selectively ejected to any of the four ejection sites. There are therefore six possible selected ejection destinations from each site, including non-ejection, permitting up to 11 different cell sub-populations to be sorted (9 channels plus two substrate surfaces); alternatively the channels may be used to sort nine different types of cells or cell sub-populations, and the vertical ejection of some of these cells onto array sites on a substrate surface, such as well plate wells may be performed simultaneously for characterization of the sorted cells.

One common task of cell selection involves colony sampling devices which stab agar surfaces containing bacterial cells or cells from other micro-organisms. Typically, an optical system drives a robotic arm containing an inoculation loop or needle. The optical system locates a colony of interest, and the needle stabs the agar and delivers the colony to a container for further growth. These systems are sometimes unreliable in their ability to find a colony of interest.

The needles also must be rinsed and sterilized between inoculations. The process of rinsing and sterilization leads to the deposition of carbon deposits and chemical residue which can interfere with further growth of the organism of interest. Mechanical robotic arms are also prone to failure, and capable of relatively imprecise positioning for sampling closely spaced colonies or delivering cells into dense arrays.

Acoustic ejection of cells directly from colonies growing on the cell surface offers a superior method for ejection of a specific number of cells from any number of colonies of bacteria growing on an agar or other semisolid or gel or the like. Densely packed colonies can be individually sampled without contamination of a sampled colony by cells from nearby colonies because of the ability to precisely and accurately focus the acoustic energy.

The presence of the colonies may be detected by acoustic microscopic means, e.g. by detecting a different acoustic impedance at the agar surface in a region having a colony compared to a region having no colony. The ability to interchange or add myriad other detection means, including standard optical microscopy and detection of intrinsic tryptophanyl fluorescence will immediately be evident.

Cells may be ejected into the wells of well plates or other physical containers. Alternatively, a planar substrate with or without specific means for attaching cells to the substrate may be employed. The containers or wells may contain nutritive media, for example nutritive agar, prior to ejection of cells thereon, or nutrients may be added after ejection. Adjusting the power or acoustic energy delivered in unit time (to a focal point sufficiently near the surface for ejection to occur, and holding this distance constant), and consequently droplet volume, permits deposition of a desired number of cells per target container or receptacle in a reproducible manner. Where multiple discrete colonies are detectible on one or more culture containers, cell samples may be arrayed according to colony onto a well plate or other substrate surface.

Depending upon the organism and morphology of the colony, the cells may be ejected from the agar or other nutritive surface without effecting specific conditions to facilitate ejection such as reducing the viscosity of the fluid in the colony or of the underlying gel or semisolid forming the substrate or medium. Some circumstances will require means for promoting specific conditions that permit ejection. Various means for effecting ejection permissive conditions include deposition of chemical or biochemical reagents at the colony sites to affect intracellular adhesion and/or viscosity of the extracellular fluid of the colony or underlying agar or gel like medium. For example a fluid containing agarose (or another agar degrading enzyme) may be deposited to liquify the agar underlying a colony, or reduce the viscosity of a liquid medium in order to facilitate ejection of cells from the colony. Other enzymes, for example, may be employed, depending upon the type of medium upon which the cells are grown.

Although eukaryotic cells are not typically grown on gel like media, they will often require some treatment to reduce intracellular adhesion, which also may be required for some prokaryotic cells. If eukaryotic cells are grown on gel or semisolid media, effecting a phase change in the substrate underlying cells by spatially circumscribed delivery of acoustic or other energy can be used in conjunction with any treatment required to reduce adhesion between cells.

Preferably the region underlying the colony is heated to a temperature that melts (Tₘ) the agar, or other gel or semisolid medium without affecting viability of the cells in the overlying colony. To this effect, a low Tₘ agar or gel like medium may be utilized. Also the phase change must be localized to the region underlying the colony from which cells are to be ejected in order that neighboring colonies are not disrupted. Wholesale melting of all the medium in an agar plate containing numerous colonies would be undesirable because all the discrete colonies would coalesce before some cells from each could be ejected. In order that cells may be ejected from each colony in rapid succession, the localized phase change or melting of the media underlying the colonies from which the cells are successively ejected must be achieved rapidly. Various means of rapid localized heating may be employed. For example an electric heating element comprising a thin member or pin can be inserted under the colony and the underlying medium melted by an electrical pulse.

Heating means that do not require physical contact between the heating device and medium are preferable. For example directed electromagnetic energy such as directed microwave or infrared radiation or a laser beam having an appropriate cross section and frequency, may be employed. Preferably the source of electromagnetic radiation is located so that the electromagnetic waves must pass through the medium underlying a colony from which cells are to be ejected, e.g. the source is located under the medium, so that the underlying medium is heated earlier and to a greater extent than the cells in the overlying colony to shield the cells from undesired heating.

Liquefying the medium beneath a colony by focused acoustic energy is a most preferable means of effecting localized melting of the medium underlying a colony from which cells are to be ejected because the depth as well as the breadth of the volume to which thermal energy is delivered can be controlled. Focused acoustic energy can be used to heat a cylindrical region having a diameter of as little as about 20 μm and height of as little as about 200 μm, without significant heating outside the cylindrical area for substances which have moderate or better thermal conductivity. Thus once a colony is located, for example by acoustic means, the focus of the acoustic energy can be adjusted so that the power is insufficient to deliver the threshold energy to eject a droplet from the surface. The region of heating is controlled in dimension in a plane parallel to the medium surface (breadth) to be wholly underlying the boundaries of the colony of interest. The depth of acoustic focus and of heating is adjusted to be below the surface or interface between the medium and overlying colony, thereby further preventing ejection. To effect more uniform heating at the desired focus without ejection, the frequency of the acoustic wave may be reduced relative to the frequency used for ejection. The acoustic wave amplitude may be adjusted to adjust heating rapidity.

In some cases the cells forming the colonies to be ejected may be transformed to liquefy the underlying medium. For example, where an agar based gel medium is used, cells may be transformed to release agarase, an enzyme which hydrolytically liquefies the underlying agar Analogous enzymes may be used for different media, for example cellulase can be used to hydrolyze various polysaccharidic moieties. The transformation to release agarase can be done solely for facilitating ejection from an agarose gel material, or it may be done in conjunction with another transformation to selectively facilitate ejection only from colonies that have been transformed. For example, bacteria may be transformed with a construct for expression of pancytokeratin (a mammalian protein) in the cytoplasm and release of agarase to the cell surroundings so that ejectability is a marker for the transformed cells.

Although not required for the methods and systems for sorting and arraying cells of the instant invention, the preferred serial and parallel multiplexing of detection and ejection lend themselves to, and are preferably integrated with a processor. The processor functions to integrate the various detection data and calculate the time that a detected and measured cell will arrive at an ejection site, and to effect the appropriate ejection, rendering the ejected droplet contained cell with the appropriate velocity vector and trajectory to correctly target the target container or channel or array site. Maximum efficiency, and throughput can be thus effected with a high level of both serial and parallel multiplexing of detection and ejection sites, with a large number of selectable ejection targets at each site.

The ability to measure a property as an ejection criterion, in addition to permitting the invention to be used for cell sorting, permits the sorting of non-living solids, gels and fluid regions discrete from the carrier fluid. It will be readily appreciated that the ejection of, for example, beads used for solid phase combinatorial synthesis and bearing some marker or property identifying the combinatorial sequence may be separated by the method of the invention.

### EXAMPLE 1

Acoustic Ejection of Monocytes onto a Substrate as an Array from a Mixture of Cells from Peripheral Blood with Concurrent Separation of Red Blood Cells, Granulocytes and Lymphocytes into Channels

Rabbit polyclonal-Ab against human MHC (displayed on all cells) is generated and a single clone is selected which binds a MHC epitope common to all humans rather than to the epitopes specific to individuals. A substrate is functionalized with the mAb by routine methods, monocrystalline Si is chosen as substrate because of the plethora of known methods for functionalizing Si.

A channel as illustrated in FIG. 6 having a 25 μm width is utilized to reduce the time spent searching for cells to eject. This central ejection channel component of the sorting unit is about 6 cm length, open on top between about 2.75 and 3.25 cm for the last 0.5 cm. The blood cells are supplied from fluidically connected channels, not shown. Two detectors, D₁ and D₂ are deployed at about the first 0.5 cm of the depicted channel. The focused acoustic energy transducers are located directly beneath ejection sites in the open on top regions of the ejection channel. To each side of each ejection site, a common fluidic channel divides into two channels that loop towards the ejection channel such that one flows parallel and the other antiparallel to the fluid flow in the ejection channel for a short distance, and sufficiently close to permit a cell to be ejected from the ejection channel to a selected target channel abutting the ejection channel near the ejection site. In all there are four target channels per ejection site, eight in all. The acoustic ejection can impart a zero magnitude velocity component, or a non-zero directional velocity component parallel to the fluid surface, e.g. in any horizontal direction. This permits cell containing droplets to be acoustically ejected, based upon detected properties, to any of the four target channels or onto a substrate surface oriented substantially parallel to the fluid surface above the ejection site, or not at all. These channels are fabricated of an HF etched glass plate heat fused to a cover glass plate (except where open on top) by routine microfabrication techniques.

The detectors employed are D₁, laser/intrinsic fluorescence, D₂ acoustic imaging. The acoustic ejection transducers also perform some detection functions at the ejection site, at a minimum detecting whether the cell is sufficiently close to the fluid surface for ejection. Cells are forced to the surface by a physical ramp like structure as depicted in FIG. 5D. Added stringency is effected by adjusting the acoustic energy delivered according to the volume of the cell to be ejected, precluding cells substantially larger than the cells sized by the detectors from being ejected if there is a mistake in sizing that substantially underestimates cell size.

Fluorescense, light scatter and acoustic data are inputted to a processor which controls the process. Sizing data including dimensions, volume of cells and detected nuclei, pertinent cytoplasmic/total/nuclear size or volume ratios are obtained from integrated acoustic and fluorescense and/or scattering data. The intrinsic Trp fluorescense emission spectrum is also measured for each cell. The decision tree is based on sizing and ratios first, and intrinsic fluorescense data second, as the majority of cells will be distinguishable by morphological characteristics. Red Blood Cells (RBCs) will have some overlap in their larger dimension with small lymphocytes, but will have a much smaller total volume even if the radii are identical because lymphocytes are spherical while RBCs are doughnut shaped. RBCs will also be non-nucleated. Small lymphocytes will have large nuclear to cytoplasmic (and nuclear to total cell volume ratios), as will medium and large lymphocytes. Medium and large lymphocytes will overlap in size with granulocytes and small monocytes, but will have substantially larger nuclear to cytoplasmic volume ratios than either, making employment of fluorescence spectrum data unnecessary except for added stringency in most cases. Monocytes that overlap in size with granulocytes will tend to have different morphological characteristics including a larger nuclear to cytoplasmic volume ratio and continuous nuclear signal, their bibbed nucleus appearing almost spherical; granulocytes will have discontinuous nuclear signal and thus appear to be multinucleate because of their highly lobulated nuclear morphology. The fluorescence spectrum will provide the conclusive data for some small monocytes for ascertaining that they are not granulocytes or large lymphocytes. Granulocytes, including PMNs, eosinophils and basophils are morphologically similar and thus distinguished based upon differences in their intrinsic Trp fluorescence emission spectra, which are characteristically shifted as a result of their different characteristic granules. Platelets are also present in peripheral blood and are technically cell fragments, non-nucleated, and smaller than RBCs. Because they are of use in surgical procedures, they are not ejected from the central or ejection channel and are collected for further purification with the blood plasma.

The peripheral blood separated may be from an individual or from a number of individuals, although, as will be readily appreciated Igs must be removed from the blood before mixing different antigenic blood types. The eight target channels at the two ejection sites are used for the different ejected cells, with small, medium and large lymphocytes, and excess monocytes ejected at the most distal ejection site to separate ejection channels. At the ejection site proximal to D₁ and D₂, PMNs, eosinophils, basophils and RBCs are ejected to separate target channels. The proximal site is also used to create an array of monocytes for experimentation, using the substrate provided. It will be readily appreciated that an additional array of any single or set of cell types may be simultaneously made at the distal ejection site, for example an array of all the nucleated cell types where no neighbor is the same cell type, or an array of large lymphocytes, which are more likely to be memory lymphocytes.

The channel is fluidically connected by routine methods to a fluid column to which the cell suspension is added. The dimensions of the column allow 5 ml of fluid carrier and cells to be added so that a sufficient column pressure exists to initiate fluid flow through the channel to allow fluid to reach the open top area in a sufficiently short time, after which the top of the column is connected to a pressure regulator which allows the gas pressure above the carrier fluid in thee column to be regulated to permit fine adjustment, termination and reinitiation of the carrier fluid flow through the channel.

The carrier fluid may be a physiologic saline or other electrolyte solution having an osmolality about equivalent to that of blood serum. The monocytes are spotted onto a substrate maintained at about 38° C. The substrate employed is planar, and the density of 10,000 sites/cm² is chosen, with each site occupied by a single cell. Circulating monocytes from 10 different individuals are obtained and purified by routine methods.

The monocytes of each individual are attached to the array by acoustic ejection of a droplet having a volume of about 4.2 pL in a pattern. Specifically, every tenth site of each row is spotted with monocytes from one individual, and the deposition of that individual's cells is staggered in subsequent rows to permit more separation between cells from an individual. Separation of an individual's cells is preferable because it provides an internal control against variation in conditions between different substrate areas. The monocytes from the remaining individuals are spotted onto the array sites in acoustically ejected droplets. Ten duplicate arrays are made.

Because monocytes are attracted by chemotaxis into inflamed tissues (where they are transformed into macrophages under the influence of immune mediators), the arrays are studied by immersing them in various physiologic solutions containing one or more inflammatory mediators, such as histamine, interleukins (Ils), granulocyte macrophage colony stimulating factor (GM-CSF), leukotrienes and other inflammatory mediators known in the art, as well as conditions which might affect inflammation, such as heat, and known antiinflammatory agents including steroids, non-steroidal antiinflammatory drugs, and random substances or those suspected to affect the activation of macrophages. It will be readily appreciated that certain mediators and combinations thereof will have a pro- or anti-inflammatory effect, and that there will be differences between individuals and to a lesser extent between individual cells. Because the monocytes are attached by the mAb/MHC specific attachment, the array will not be disrupted by immersion.

The transformation of the monocytes into macrophages and of macrophages back to monocytes may be observed by light microscopy without afecting cell viability. Other known methods of measurement of individual cells include XPS (X-ray photoelectron spectroscopy) of individual cells. Because immune cells, especially activated macrophages are able to activate immune cells by release of immune mediators and chemotactic agents, the possibility exists that one individuals monocytes are not responsive to an immune mediator or condition, but responsive to the immune mediators released by another individual's macrophage which was responsive to the experimental condition. To control for the preceding, standard well plates are used as controls using the identical method, with multiple monocytes from the same individual in each well (for 96 well plates, 9 wells/individual, 110 cells each). A final control using well plates without the mAb/MHC attachment system is also created by the method described, surface tension sufficing to hold the ejected cell containing droplets in place. It is readily appreciated that the 110 droplets deposited in each well plate are preferably deposited at different locations within the well to prevent the formation, by multiple deposition, of droplets too big to be held in place by surface tension.

The high throughput design depicted in FIGS. 7A and 7B, and described in the foregoing may also be employed in substantially the same manner as used in this example with the configuration depicted in FIG. 6. One advantage of this design is that cells are inherently recirculated. In some instances an ejection site might be overwhelmed by the number of cells which must be manipulated. In this example this is especially applicable to RBCs which are the most numerous cells. The ability to overcome this problem by using more than one ejector for the ejection of the RBCs, or adding a third RBC dedicated ejector to the system, as embodied in either the system illustrated in FIG. 6 or FIG. 7, will be readily apprehended. Recirculating RBCs which are not ejected in a first pass in order to permit the orderly procession of cells without impaction and disruption of flow, can be effected by recirculating the carrier fluid after all the less numerous cell types have been sorted. The embodiment depicted in FIG. 7A & FIG. 7B is especially suited for such recirculation.

### EXAMPLE 2

Bronchoalveolar Lavage Human Airway Epithelium (HAE) Cell Array for Studying Inflammatory Response with Simultaneous Cell Counting

The method of the preceding example is adapted to arraying HAE cells obtained from bronchoalveolar lavage with simultaneous sorting and differential cell count. In addition to epithelial cells, bronchoalvolar lavage fluid routinely contains other cells. Cells found in lavage fluid include the agranulocytic leukocytes, lymphocytes and monocytes, which are typically activated as macrophages, and granulocytic leukocytes, neutrophils (PMNs), eosinophils and basophils. Often present are pathogens such as viruses, including influenza viruses and DNA viruses, including herpesvirus family members, most notably CMV (cytomegalovirus) and KSV (Kapsoi sarcoma associated herpesvirus), fungal species, including Cryptococcus albidus, Coccidioides immitis and Aspergillus flavus, and eukaryotic opportunistic pathogens such as Pneumocystitis carinii, which is found in healthy patients and causes pneumonia in the severely immunocompromised, in addition to the prokaryotes or bacteria, including the members of ubiquitous gram positive and negative bacteria groups, Mycobacteria species, and obligate intracellular prokaryotes, chlamydia, mycoplasma and rickettsia. With the possible exception of the obligate intracellular prokaryotes, all the pathogens may be cultured by routine microbiological and virological methods from the fluid remaining after all mammalian cells have been ejected. Pneumocystitis carinii cysts (d≈5-7 μm), trophozoites or sporozoites may be ejected for staining as may be extracellular competent (non-obligate intracellular or extracellular) bacterial species (typical d≈1 μm) and directly stained and examined instead of or in addition to culturing as required for identification. Pneumocystitis carinii cysts, for example, are identifiable without culturing by microscopic examination of stained specimens.

The sorting, counting and arraying proceeds substantially as described in Example 1 with the additional recording of the identity of each cell ejected for counting purposes. Often differential counts alone will provide useful diagnostic and pathophysiologic information. For example, elevated eosinophils and lymphocytes will indicate asthma or related eosinophilic lung inflammatory processes. Separated lymphocytes may be further ascertained to have elevated activated T lymphocytes expressing cell surface activation markers HLA-DR, IL-2R (interleukin 2 receptor) and VLA-1. Alternatively the lymphocytes can be arrayed onto a substrate functionalized at different sites with antibodies that recognize the preceding markers mentioned. Fibrotic inflammatory disease of the lower airways, termed generally interstitial lung disease will exhibit a predominance of PMNs and alveolar macrophages. Immunocytochemistry of macrophages, and to a lesser extent PMNs and airway epithelial cells demonstrates these cells to contain characteristic cytokines, for example IL-1β, IL-6 and IL-8 in chronic lung disease of prematurity (Kotecha, et al. (1996) Pediatri Res: 40:250-56). Bacterial pneumonias exhibit similar differential cell counts, but with more immune cells and bacteria particles present in the lavage fluid and sometimes visible within macrophages, and are thus distinguishable.

Using the differential cell counts and microbiological/virological pathogen culture and identification methods, eosinophilic and neutrophilic primary inflammatory processes are distinguished from one another and inflammations secondary to infectious processes in the patients from which lung lavage samples are taken. HAE cells from the patients are also studied in the arrays.

As is readily appreciated, a channel having appropriate dimensions must be provided (just larger than the HAE cells and possibly large monocytes, thus approximately 25-30 μm). Alternatively the width of the channel is just wider than the cells, but to permit faster loading, the depth is approximately three times the diameter of the cells and a ramp as depicted in FIG. 5D is employed in the channel flow path just prior to the channel region which is open. Alternatively a photon field as may be provided by a laser as commonly used in optical tweezers may be employed to force the cells close to the surface. Arrayed HAE cells are obtained by broncoalveolar lavage, and ejected onto the substrate surface during sorting and counting as described herein and in the preceding examples. Before being loaded for ejection the lavage fluids are treated to suspend adhering cells as individual cells by disaggregating them by conventional tissue culture methods.

Experiments on HAE cells may be conducted under conditions which do permit cell division. The need for the preceding as well as the conditions required for this will be appreciated by one of ordinary skill. The controls with well plates are useful but not as critical as with the monocytes.

### EXAMPLE 3

HAE Cell Array for Studying Individual Susceptibility to Mutagenesis as a Proxy for Carcinogenesis

The method of the preceding example is adapted to permit exposing the arrayed HAE cells to chemical and other mutagens such as heat and radiation. Genetic damage is measured at different times after the exposure is discontinued by routine methods for biochemical assaying of broken crosslinked and otherwise damaged DNA. Differences in DNA repair enzyme genetics may be studied by comparing recovery (extent of reduction of damage) at various times after exposure. The well plate arrays remain useful as controls, and cells may be cultured in the well plates or array cells may be removed and cultured to determine the actual appearance of dysplastic or neoplastic cells in subsequent cell generations after the exposure, and the extent of any dedifferentiation in any dysplastic or neoplastic cells detected.

### EXAMPLE 4

Cell Patterning

The method of Examples 1 and 2 is adapted to pattern basal squamous cells. Basal squamous keratinizing epithelial cells and squamous non-keratinizing epithelial cells are patterned on a nitrocellulose substrate functionalized as in Example 1. The pattern generated emulates the vermillion border of the lip. The patterned cells on substrate are then immersed in suitable culture media, and studied for forming a skin/non-keratinizing junction.

### EXAMPLE 5

Acoustic Ejection of Lymphocytes from Blood onto an Epitope Array

Small, medium and large lymphocytes are ejected by the methods of the preceding examples to form a clonal epitopic array. Two different dimension channels appropriately designed to force the cells near the surface are constructed side by side. The wider channel is about 15 μm wide for medium and large lymphocytes; the narrower channel is 10 μm wide for small lymphocytes. Small lymphocytes may be separated from large and medium lymphocytes by routine methods, or by acoustic ejection. An amount of energy barely sufficient to eject small lymphocytes is applied with all lymphocytes in the mixture passing through one common channel (15 μm wide). The energy is applied to each lymphocyte which is detected at the channel opening or aperture which forms the ejection region. The ejected lymphocytes may be ejected onto a substrate and washed into a petri dish or other container. Alternatively, the acoustic energy can be delivered to eject the droplet in a non-vertical trajectory so that the droplets land in a nearby container, such as a channel that is open on top sufficiently near the ejection channel.

The epitope array is a combinatorial tetrapeptide array formed from naturally occurring amino acids. Other epitopes are readily appreciated to exist both in proteins as a result of non-primary structure and from peptidic molecules bearing haptens or other biomolecules such as peptidoglycans or polysaccharides. Thus only a small fraction of the approximately 10¹² epitopes will be arrayed. Both T and B cells will bind these epitopes, by slightly different mechanisms as will be readily appreciated. The tetrapeptide arrays can be made by various methods, for example by adaptation of solid phase peptide synthesis techniques to the focused acoustic ejection of reagents as described in the copending application on combinatorial chemistry described above. As 1.6×10⁴ different natural tetrapeptides exist, 16 array synthesis areas, each 1 cm², must be made to make all the tetrapeptides and maintain appropriate density for allowing separation of individual cells.

Cells are spotted onto the array sites as rapidly as possible (thus two channels for maintaining single file lines of cells in the channels despite the different sizes). When each array site (all 16,000 sites) has had a droplet ejected onto it, the arrays are washed to remove cells that do not bind the epitope at the site of deposition. The arrays are imaged to determine which sites bind a cell, and the cycle is repeated for sites not binding a cell, which are re-spotted. Immediately apprehended is that this process requires imaging of the array after washing, and overall must be automated. Automation of such a system is readily attainable, and invaluable information and clonal separation would be derived prior to completion of the project. Use of different types of epitopes would further extend the cataloguing.

### EXAMPLE 6

Ejection of Bacteria to Select Transformed Bacteria

E. coli are transformed by routine methods to express pancytokeratin, a eukaryotic protein, by a construct that also causes expression and display of streptavidin on the cell surface. Using a substrate biotinylated by routine methods, the transformed cells are selected by acoustic ejection onto the substrate. All E. coli cells are deposited onto the substrate by acoustic ejection as described in the preceding Examples 1-5. The ejection channel size may be adapted to bacterial dimensions (1 μm) but this is attainable by known microfabrication methods. Transformed cells will be specifically bound to the biotin cognate moiety by the marker moiety, streptavidin. Washing the substrate will remove cells that have not been transformed, leaving only transformed cells attached to the substrate.

The preceding ability to separate transformed from untransformed bacteria is combined with the ability to remove all blood cells from peripheral blood in Example 1 to comparatively evaluate the ability of transformed and untransformed E. coli to cause bacteremia in mice and to compare the immune response mounted against the transformed and untransformed bacteria. The blood of inoculated mice is drawn and sorted as in Example 1, except that in addition to sorting all the different blood cells, the number of cells of each type are counted to provide information as to immune response. Baseline counts are done by routine methods prior to inoculation. After sorting and counting of blood cells according to Example 1, only bacteria, platelets and plasma remain in the central ejection channel. Although roughly the same size and geometry as the platelets the bacteria can be distinguished from the platelets by detecting the nucleoid, where the bacterial chromosome is localized by light scattering or other means, or by use of intrinsic Trp fluorescence, which will differ between platelets and bacteria. All bacteria are counted and ejected, and the number and fraction of ejected bacteria that are transformed is determined by counting those bacteria that are attached to the biotinylated substrate surface after it is washed.

Four groups of mice are evaluated. The first group is inoculated intravenously with a placebo inoculation of an appropriate carrier, such as buffered saline, having no bacteria and equal in volume to the inoculation volumes for the other groups. The second group is inoculated with an equal volume of carrier containing a known number of transformed, live E. coli, as a standardized number of bacterial cells per volume. The third group is inoculated with an equal volume of carrier containing a known number of non-transformed, live E. coli of the same strain as the transformed bacteria, as a standardized number of bacterial cells per volume. The fourth group is inoculated with an equal volume of carrier containing a known number of live E coli the bacteria being all of the same strain, the population being a mixture of ½ transformed bacteria and ½ non-transformed bacteria, as a standardized number of bacterial cells per volume. Blood is drawn from the mice at regular intervals after the inoculation for one week or until death of the mice from bacteremia. Statistical data on cell type population and differential count from all groups will also provide data on individual variation of immune response within groups.

Data from the first group will primarily be used as a control for determining the spontaneous entry of bacteria into the blood of non-inoculated mice, whether displaying streptavidin or not; all bacteria detected in the blood of mice from this group will be further cultured and characterized for control purposes. Data from the second group, in addition to being a control for the fourth group can be compared to data from the third group to study relative pathogenicity without competition from non-transformed bacteria. Additionally data from the second group can be used to study loss of all or part of the construct, e.g. those bacteria obtained from group 2 mice after inoculation that do not display streptavidin and bind the biotinylated surface may be cultured and immunostained to determine whether they are expressing pancytokeratin to quantify reversion for control purposes. Data from the third group can also be obtained for determining whether spontaneous transformation to pick up the displayed streptavidin, and the remote possibility that the streptavidin/pancytokeratin construct has (somehow) entered that population. The fourth group provides data on the ability of the transformed and untransformed strains to cause bacteremia under competitive conditions. Data from the fourth group is compared for total bacteria per volume with the other groups. Also the relative proportions of transformed and non-transformed bacteria may be analyzed after appropriate consideration of spontaneous infection, or loss or gain of transformation. For these purposes, transfer of the transforming construct by conjugation is not considered spontaneous. The possible addition of other groups with different inoculation proportions of transformed and non-transformed bacteria will be readily appreciated.

### EXAMPLE 7

Ejection of Cells Directly from Colonies Growing on Agar Medium

One mode of accurate, contactless cell selection of colonies on agar is provided by focused acoustic energy to effect droplet ejection. The ejected droplets may contain one or more cells, and may be adjusted in volume to deposit more or fewer cells per ejection. A colony of cells is sampled from the center in the plane parallel to the surface of the medium or substrate to avoid contamination of the sample by organisms from neighboring colonies. The number of separate samples from an individual colony that may be thus deposited depends on colony size and sample size; at minimum, several samples of even the smallest colonies can be ejected.

A routine throat smear is cultured on standard blood agar medium in a conventional plastic petri dish, and the culture is incubated at about 38° C. for 72 hours. After the incubation, the acoustic transducer is placed under the plastic petri dish containing the agar and bacterial colonies, and the presence or absence of colonies is detected via acoustic microscopy.

The same acoustic transducer used to locate the cells is used to propel the cell from the surface of the agar, provided that the surface has the correct viscosity. Focused acoustic energy is delivered immediately beneath the colony center at a focal point for thermal delivery about 75 μm beneath the surface of the agar medium. The pulse of acoustic energy has sufficient power and a sufficient duration to liquefy a cylinder of agar having dimensions in the plane parallel to the medium surface that are within the dimensions of the colony in this plane and extend in the direction perpendicular to the surface plane to the substrate surface which liquefies at temperatures close to about 45° C. (Gibco, Inc, now Life Technologies, Rockville Md., a division of Invitrogen,). The need to calibrate the thermal delivery acoustic pulse to the specific agar composition and depth, and petri dish to melt cylindrical volumes of various diameters will be immediately appreciated. Alternatively, a scanning laser may be used with the low melting agar. Utilizing a low-melt agar permits surface liquefaction without significant reduction in the viability of the selected micro-organisms on the agar surface. If a laser is employed, the laser placement can be coupled to the colony location determined by acoustic microscopy. The focal point of acoustic energy for ejection is at the surface of the medium. By locally heating the agar, the viscosity at the surface of the medium is reduced to allow ejection of the colonies of interest directly into a well plate or other container of interest.

Acoustic delivery of thermal energy is used to effect the local melting beneath colonies prior to acoustic ejection. In this manner, each colony is sampled four times. Two duplicate arrays of cells ejected from bacterial colonies are made using standard well plates containing nutritive agar medium, with droplets having a volume of about 0.1 to 1.0 pL. That the different wells may contain different medium and nutrients will be immediately apprehended. Two additional samples each having a volume of about 1.0 to 100 pL (in multiple droplets as required) from each colony are deposited onto a clean surface and washed using saline into a flask containing nutritive fluid (or alternatively into flowing fluidic channels that empty into containers of nutritive fluid).

The sampled cells are immediately cultured on petri dishes from the flasks, by conventional methods of cell culture. The array plates and flasks are stored chilled to slow bacterial reproduction, permitting future culturing and testing. The original culture petri dish is also stored chilled pending culture results. The culture results from the specific throat culture are examined by conventional microscopy and other means. Numerous gram negative and gram positive bacterial species are initially identified, as well as several yeast species, all non-pathogenic to immunocompetent adult humans. Further culturing from the flasks using nutritive agar media containing antibiotics by routine methods for determining antibiotic resistance reveals that different colonies of the same species of bacteria have different antibiotic resistance, demonstrating the different colonies to be different strains or sub-strains.

The method of ejecting cells from colonies growing on agar medium may be used to selectively eject transformed cells. An indicator is used in the transforming construct along with the desired genetic transformation, here expression of pancytokeratin. For example the construct can additionally transform the cells to secrete agarase, and the transformed colonies selected by detecting an altered acoustic impedance. Alternatively, transformed colonies may be selected optically if the construct is designed to cause transformed cells to co-express a marker such as green fluorescent protein. Only green flourescent colonies detected optically are ejected.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains. All patents, patent applications, journal articles and other references cited herein are incorporated by reference in their entireties.

We claim:

1. A separation method comprising the steps:
(a) detecting in a fluid having a surface and a plurality of localized volumes having a different acoustic impedance than the fluid a single localized volume located sufficiently near the surface for ejection;
(b) determining whether the single localized volume possesses one or more properties;
(c) selecting the single localized volume for ejection from the fluid based on the determination of one or more properties in step (b);and
(d) ejecting the single localized volume from the fluid by use of focused energy.

2. The method of claim 1, wherein the focused energy is focused acoustic energy.

3. The method of claim 1, wherein the focused energy is focused electromagnetic energy.

4. The method of claim 1, wherein the localized volume comprises a solid or gel particle.

5. The method of claim 1, wherein the localized volume comprises a cell.

6. The method of claim 5, wherein the localized volume comprises a living cell.

7. The method of claim 1, wherein the localized volume is ejected in a trajectory substantially perpendicular to the fluid surface.

8. The method of claim 1, wherein the localized volume is ejected in with a velocity component perpendicular to the fluid surface and a velocity component parallel to the fluid surface to effect a trajectory whereby the localized volume experiences a net displacement in a direction parallel to the fluid surface.

9. The method of claim 8, wherein the trajectory is directionally controllable, whereby the direction of net displacement parallel to the fluid surface is thereby directionally controllable.

10. The method of claim 8, wherein the non-vertical distance of travel parallel to the fluid surface is controllable by varying the focused energy.

11. The method of claim 9, wherein the non-vertical distance of travel parallel to the fluid surface is controllable by varying the focused energy.

12. The method of claim 11, wherein the determining in step (b) of the one or more properties is a quantitative or semiquantitative determination and the selecting of step (c) is between non-ejection and multiple ejection trajectories, the selecting depending upon the quantitative or semiquantitative determination.

13. The method of claim 12, wherein the fluid is contained in a fluidic channel.

14. The method of claim 13 wherein data from said detecting of (a) and said determining of (b) is inputted into a processor, whereby the processor directs said selecting of (c) and said ejecting of (d) by reference to the measured data, and programmed selection criteria and system parameters.

15. The method of claim 1 wherein the localized volumes are circumscribed volumes comprising living cells.

16. A system for the separation, from a carrier fluid having a fluid surface and containing a plurality of circumscribed volumes having a different acoustic impedance than the carrier fluid, of one or more of the circumscribed volumes, the system comprising:
a fluidic container;
a detector for detecting the localized volume and determining a property of the localized volume; and
an acoustic ejector of fluid droplets from the carrier fluid, comprising an acoustic radiation generator for generating acoustic radiation and a focusing means for focusing the acoustic radiation at a focal point near the fluid surface;
wherein the circumscribed volumes present in the carrier fluid that are detected to be sufficiently near the fluid surface for ejection may be acoustically ejected to a target from the carrier fluid in a fluid droplet depending upon whether the circumscribed volume possesses one or more properties.

17. The system of claim 16 further comprising a means for positioning the ejector in acoustic coupling relationship to the container in an appropriate position to permit the focusing means to focus the acoustic radiation at the focal point in a desired point of the carrier fluid.

18. The system of claim 17 further comprising a processor for integrating said detector, said acoustic ejector, and said means for positioning said acoustic ejector with respect to said container to eject the circumscribed volumes to selected targets based upon the detected property.

19. The system of claim 18 wherein said fluidic container comprises a fluidic channel that has a surface, the fluidic channel having dimensions permitting the carrier fluid containing the plurality of circumscribed volumes to flow freely through the channel.

20. The system of claim 19 wherein said fluidic channel has dimensions permitting the plurality of circumscribed volumes or a subset of the plurality of circumscribed volumes to flow freely through the channel only in substantially single file.

21. The system of claim 19 wherein the target is an array site or a target fluidic channel.

22. The system of claim 21 wherein based upon a quantitative or semiquantitative measurement of the property by said detector the circumscribed volume is selected not to be ejected, to be ejected into a target channel or to be ejected onto an array site.

23. The system of claim 22 wherein the array site is a well plate well.

24. The system of claim 22 wherein the circumscribed volume ejected into the target channel may be ejected from the target channel into a subsequent target channel or a subsequent array site.

25. The system of claim 16 or 22 wherein said circumscribed volume is a cell.

26. The system of claim 25 wherein the cell is a living cell.

27. A system for the separation, from a carrier fluid having a surface containing a plurality of circumscribed volumes having a different acoustic impedance than the carrier fluid, of one or more of the circumscribed volumes, the system comprising:
a container;
a substrate having a substrate surface oriented substantially parallel to the surface;
means for acoustically ejecting from the carrier fluid through the aperture onto a location on the substrate surface a circumscribed volume having a different acoustic impedance than the carrier fluid,
wherein the circumscribed volume present in the carrier fluid that is detected near the fluid surface below the aperture may be acoustically ejected from the carrier fluid onto a substrate location in a fluid droplet depending upon whether the localized volume possesses one or more properties.

28. The system of claim 27 further comprising a processor for integrating said detector, said acoustic ejection means, and said means for positioning said acoustic ejector with respect to said container to eject the circumscribed volumes to selected targets based upon the detected property.

29. The system of claim 28 wherein said fluidic container comprises a fluidic channel that has a surface, the fluidic channel having dimensions permitting the carrier fluid containing the plurality of circumscribed volumes to flow freely through the channel.

30. The system of claim 29 wherein said fluidic channel has dimensions permitting the plurality of circumscribed volumes or a subset of the plurality of circumscribed volumes to flow freely through the channel only in substantially single file.

31. The system of claim 29 wherein the target is an array site on said substrate surface or a target fluidic channel.

32. The system of claim 31 wherein based upon a quantitative or semiquantitative measurement of the property by said detector the circumscribed volume is selected not to be ejected, to be ejected into a target channel or to be ejected onto an array site.

33. The system of claim 32 wherein the array site is a well plate well.

34. The system of claim 32 wherein the circumscribed volume ejected into the target channel may be ejected from the target channel into a subsequent target channel or a subsequent array site.

35. The system of claim 27 or 32 wherein said circumscribed volume is a cell.

36. The system of claim 35 wherein the cell is a living cell.

37. A method for ejecting one or more cells from a colony of cells disposed on a medium surface to a target comprising a substrate surface or a container, the method comprising locating a colony and ejecting the cells by focused energy.

38. The method of claim 37 wherein ejection is by focused acoustic energy.

39. The method of claim 38 wherein the location is by measuring acoustic impedance at the medium surface.

40. The method of claim 37 or 39 wherein the medium is a gel or semisolid, further comprising causing the medium to liquify in a volume transected by a plane parallel to the medium surface wholly underlying the boundaries of the colony of cells.

41. A system for ejecting cells from a colony of cells disposed on a surface of a medium having a bulk, comprising:
an acoustic energy source;
means for focusing acoustic energy from said acoustic energy source to a focal point;
means for positioning the focal point at any point in the medium or the colony of cells,
wherein the cells are ejected by delivering acoustic energy to the focal point with an adequate power and for a sufficient time to deliver a quantity of acoustic energy that ejects the cells.

42. The system of claim 41 further comprising a processor for integrating said acoustic energy source, said means for focusing acoustic energy, and said means for positioning the focal point at any point in the medium to eject the cells to selected targets from the cell colonies.

43. The system of claim 41 wherein the colony of cells is located by scanning the focal point across the surface to detect an area of the surface having a different acoustic impedance than the surface without any colony of cells.

44. The system of claim 41 or 42 wherein the medium comprises a gel and acoustic energy is additionally delivered to the bulk of the medium prior to cell ejection into a volume transected by a plane parallel to the medium surface wholly underlying the boundaries of the colony of cells.

45. The system of claim 44 wherein the acoustic energy is delivered to the volume at a geometric center of the volume, the geometric center located a distance beneath the surface of the medium in the bulk.

46. The system of claim 44 wherein the geometric center of the volume is located about 50 to 150 μm beneath the surface of the medium in the bulk.

47. The system of claim 41 wherein the medium in the volume undergoes a change prior to ejection from the geometric center of the volume to the surface of the medium, whereby the change reduces the quantity of acoustic energy to eject the cells.

48. The system of claim 47 wherein the change is a liquefying and the liquefying is detected by measuring a change in acoustic impedance or acoustic attenuation of the volume.

49. The system of claim 42 wherein the target is an array site or a fluidic channel.

50. The system of claim 48 wherein the liquefying is caused by deposition of a reagent, a characteristic of the cells in the colony or the delivery of energy to the medium.

51. The system of claim 42 further comprising a detector which measures a property of the colony of cells.

52. The system of claim 51 wherein based upon a quantitative or semiquantitative measurement of the property by said detector the cells from the colony are selected not to be ejected, to be ejected into a target container or to be ejected onto an array site.

53. The system of claim 52 wherein the array site is a well plate well.

54. The system of claim 52 wherein the target container is a fluidic channel.

55. The system of claim 52 or 54 wherein cells ejected into the target container may be ejected from the target container into a subsequent target container or a subsequent array site.

56. The system of claim 41, 42 or 52 wherein the cells are living cells.

57. A method for conditioning a material to facilitate subsequent acoustic ejection in a delineated volume of material comprising delivering acoustic energy to a focal point in the material.

58. The method of claim 57 wherein the material at the focal point is heated.

59. The method of claim 57 wherein the material at the focal point undergoes a phase transition.

60. The method of claim 57 wherein the material is a gel, amorphous solid or semisolid and the phase transition at the focal point is to a liquid.

61. The method of claim 60 wherein the material is an agar gel having a surface and the focal point is below the surface of the agar gel and beneath a colony of cells.

62. A method for delivering thermal energy to a delineated volume of material comprising delivering acoustic energy to a focal point in the material.

63. The method of claim 62 wherein the material at the focal point is heated.

64. The method of claim 62 wherein the material at the focal point undergoes a phase transition.

65. The method of claim 62 wherein the material is a gel, amorphous solid or semisolid and the phase transition at the focal point is to a liquid.

66. The method of claim 65 wherein the material is an agar gel having a surface and the focal point is below the surface of the agar gel and beneath a colony of cells.
